(19) United States
(12) Patent Application Publication
(10) Pub. No.: US 2025/0154040 A1
(43) Pub. Date: May 15, 2025

(54) COMPOSITIONS, SYSTEMS, AND METHODS FOR PROCESSING RECIRCULATING AQUACULTURE WATER

(71) Applicant: 683107 Alberta Ltd., Lethbridge (CA)

(72) Inventors: Jacob HØJGAARD, Lethbridge (CA); Malcolm LOWINGS, Lethbridge (CA); Michael LOWINGS, Lethbridge (CA); Camilla NESBØ, Lethbridge (CA); Peter STOUGAARD, Lethbridge (CA)

(21) Appl. No.: 18/834,932

(22) PCT Filed: Jan. 31, 2023
(86) PCT No.: PCT/CA2023/050133

§ 371 Date: Jul. 31, 2024

Related U.S. Application Data
(60) Provisional application No. 63/305,436, filed on Feb. 1, 2022.

(51) Int. Cl. C02F 3/34; C02F 1/28; C02F 3/06; C02F 3/10; C02F 101/32; C02F 101/34; C02F 103/22

(52) U.S. Cl. CPC C02F 3/348; C02F 1/281; C02F 1/285; C02F 1/286; C02F 3/06; C02F 3/104; C02F 3/107; C02F 2101/322; C02F 2101/34; C02F 2103/22; C02F 2203/004

(57) ABSTRACT

Disclosed herein are microbiome-based compositions for reducing and/or eliminating target compounds from water within recycled aquaculture systems. This disclosure also relates to methods for preparing dynamic microbiome-based compositions, and methods for deploying the dynamic microbiome-based compositions into recirculating aquaculture water systems.

### TECHNICAL FIELD

The present disclosure generally relates to aquaculture systems. In particular, the present disclosure relates to compositions, systems, and methods for processing and recirculating aquaculture water.

### BACKGROUND

Recirculating aquaculture systems (RAS) are useful for increasing production of water-based food products, such as fish and shellfish (fresh water or marine species, collectively referred to as “fish” herein). Generally, RAS include one or more tanks for holding the fish and water, either marine or fresh water. RAS further include a pumping system for circulating water from the one or more tanks to a waste removal/treatment system through a system of conduits. Typically, the waste removal/treatment system is configured to treat chemical waste (for example, ammonia, carbon dioxide) and remove solid waste from the circulating water. RAS can also be configured to introduce oxygen into the circulating water. While some water volume may need to be replenished due to evaporation and splashing loss, practically RAS may be considered a closed fluid circuit.

RAS systems are useful because they use relatively little water, they can occupy a reasonable physical footprint and, therefore, they can be an economical alternative to open water fishing or fish-farming in open water.

While the waste removal/treatment systems may be effective at reducing the levels of some waste, they are not effective at reducing levels of certain chemicals within the circulating water. For example, because RAS systems can use relatively small amounts of water, they may be prone to developing biofilms and, therefore, also prone to bacterial accumulation. Such bacteria can release one or more off-flavour compounds into the circulating water. The off-flavour compounds are then absorbed by the fish being grown. Overtime, the off-flavour compounds accumulate in the flesh of the fish, causing the product to have an unpleasant taste and/or odour.

Typical approaches to addressing off-flavour compounds include replacing the circulating water with new water, cleaning one or more tanks and conduits to reduce or remove the biofilm, and additionally, moving the fish to a new tank or RAS and placing them on a restricted diet in order to allow the off-flavour compound to be cleared from their bodies. This clearance, or depuration procedure can take days or weeks and may put further economic stress on the operator of the RAS and the altered diet may ultimately impact the market price of the fish due to changes in mass and texture of the fish, as well as increasing the water-use footprint of the operation. Other, typically less effective, approaches for dealing with off-flavour compounds include employing further filters, oxidizing agents and ultraviolet light.

A further challenge that off-flavour compounds pose in RAS is that they are difficult to detect until, at least sometimes, analysis of the fish already indicates an off-flavour or odour.

### SUMMARY

The embodiments of the present disclosure generally relate to compositions, systems, and methods for processing water of a recirculating aquaculture system (RAS). In particular, the compositions, systems, and methods disclosed herein pertain to removal from RAS water, of one or more target compounds, such as organic compounds like terpenoids and other compounds known to cause undesirable flavors of RAS farmed fish and/or shellfish. Such off-flavours typically render the farmed product permanently or temporarily unmarketable and the known methods for mitigating the impact of the off-flavour compounds can also negatively impact the farmed product. Additionally, the compositions, systems, and methods disclosed herein relate to removal of one or more steroid compounds, such as corticosteroids, from RAS water. Such steroid compounds are known to disrupt animal health and growth, which in turn can interfere with RAS operations.

Some embodiments of the present disclosure relate to compositions, methods and apparatus for reducing and/or removing of one or more target compounds from RAS water including, but are not limited to a terpene, a terpenoid, an amine-based compound, a haloanisole, a steroid such as a corticosteroid like cortisol, or combinations thereof. Further specific, but non-limiting examples of a target compound include: geosmin and 2-methylisoborneol (2-MIB). Target compounds may also include steroid compounds like the corticosteroid cortisol. Some embodiments of the present disclosure relate to compositions, methods and apparatus for reducing and/or removing two or more target compounds from RAS water.

According to some embodiments of the present disclosure, the compositions disclosed herein may comprise one or more enriched microbiome-based components and an optional biofilm that is configured to coalesce at least a portion of the enriched microbiome-based components for loading on to a carrier and/or for mixing with an additive.

According to some embodiments of the present disclosure, one or more selected microbiome components may be isolated from water that has been exposed to one or more target, off-flavour compounds, then enriched by methods disclosed herein, and maintained in fluid media supplemented with one or more target, off-flavour compounds. In some embodiments of the present disclosure, the enriched microbiome components may be combined with a selected carrier or additive, optionally by the presence of the biofilm, then deployed within a recirculating aquaculture system (RAS). The selected microbiome components may comprise complex naturally occurring mixtures of biological species collected from selected RAS, or elsewhere, and they may be further selected for their ability to consume (or otherwise chemically alter) one or more target, off-flavour compounds as energy and/or nutrient sources. According to some embodiments of the present disclosure, the selected microbiome components may be enriched with one or more target compounds, such as geosmin and/or 2-MIB and/or cortisol. Selecting the microbiome components may result in an increased abundance of the biological species that consume one or more target compounds relative to other biological species within the selected microbiome components that do not consume one or more target compounds. The increased abundance may be due to an increased amount of the consuming species, a decrease in the amount of the non-consuming or producing species or a combination thereof.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 is a schematic illustration of a process according to an embodiment of the present disclosure, for the capture and enrichment of geosmin and/or 2-methylisoborneol and/or cortisol-degrading microbiomes from recirculating aquaculture systems (RAS), for use in one or more of aquaculture feed formulations and RAS processing equipment.

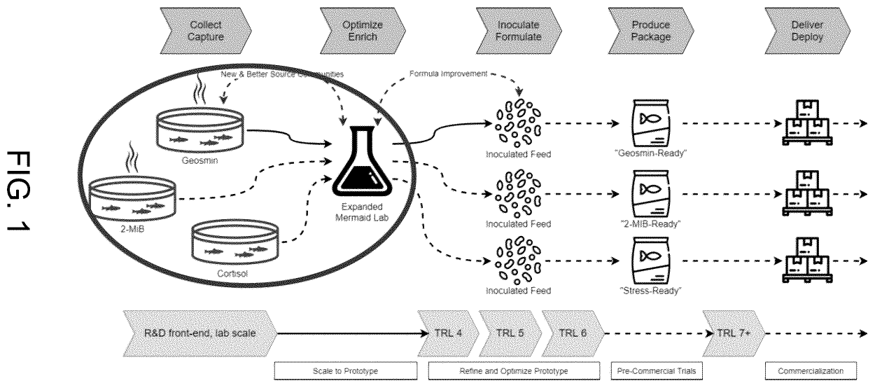

FIG. 2 is a schematic illustration of a system according to an embodiment of the present disclosure, for monitoring, assessing, and modulating recirculating water and the inherent microbial populations in RAS, by controlled selective inputting of selected enriched microbiomes produced by some of the methods disclosed herein.

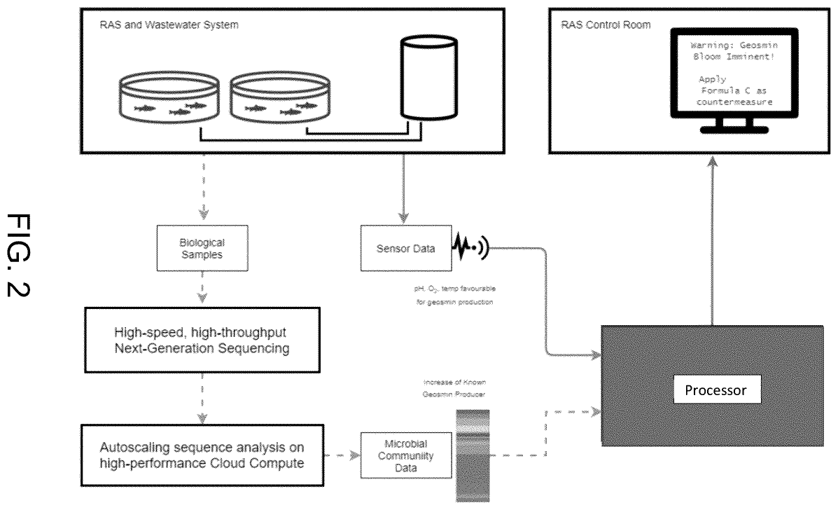

FIG. 3 is a schematic illustration of a process to collect RAS microbiome samples from recirculating aquaculture water, to select, to enrich geosmin-degrading microbial species in the RAS microbiome samples, to formulate aquaculture feed compositions or carriers that are inoculated with the enriched consortia.

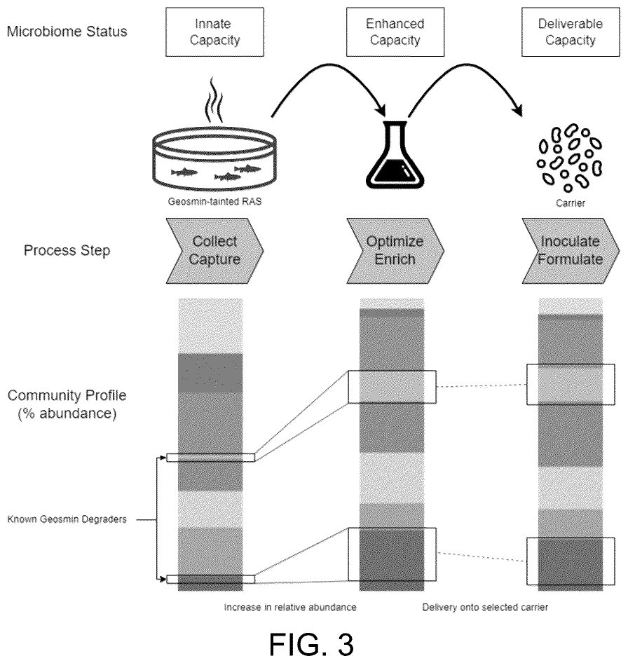

FIG. 4 is a chart showing microbial growth measured as changes in optical density (OD) at 600 nm, in geosmin-amended aquaculture water (0.5 ml of 1% geosmin added to 500 mL Buschnell Haas minerals (BH)—amended water) during enrichment of microbiomes sampled from a RAS.

FIG. 5 is a chart showing microbial growth measured as changes in optical density (OD) at 600 nm, in geosmin-amended aquaculture water (0.5 ml of 1% geosmin added to 500 mL BH-amended water) during enrichment of microbiomes sampled from a RAS.

FIG. 6 is a chart showing microbial growth measured as changes in optical density (OD) at 600 nm, in a first set of aquaculture water samples (AS) taken from a first facility in northern Europe, as amended with selected concentrations of geosmin.

FIG. 7 is a chart showing microbial growth measured as changes in optical density (OD) at 600 nm, in a second set of aquaculture water samples (BH) amended with selected concentrations of geosmin.

FIG. 8 is a chart showing microbial growth measured as changes in optical density (OD) at 600 nm, in the BH aquaculture water amended with selected concentrations of geosmin.

FIG. 9 is a chart showing the relative amounts of geosmin in the different enrichment treatments, normalized to a benzaldehyde-d5 internal standard.

FIG. 10 is a chart illustrating microbial population community diversity development determined with 16S rRNA gene amplicon analyses over a 50-day enrichment period in AS aquaculture water and BH aquaculture water amended with selected concentrations of geosmin. The different colours and shades of colours designate unique amplicon sequence variants (ASVs).

FIG. 11 is a colour-coding bar chart identifying the individual ASVs detected and illustrated in FIG. 10.

FIG. 12 is a chart showing a Principal Components Analysis (PCA) of ASVs detected in AS aquaculture water samples, in geosmin-amended AS aquaculture water samples, and in the controls. Prior to the analysis, ASV's that were not present in more than 0.1% relative abundance in any sample, were removed. The data were initially processed by applying the Hellinger transformation. The relative contribution (eigenvalue) of each axis to the total inertia in the data is indicated as “%” in the axis titles.

FIG. 13 is a chart illustrating microbial population community diversity development determined with 16S rRNA gene amplicon analyses of cultures sampled from BH aquaculture water amended with selected concentrations of geosmin. The different colours and shades of colours designate unique amplicon sequence variants (ASVs).

FIG. 14 is a colour-coding bar chart identifying the individual ASVs detected and illustrated in FIG. 13.

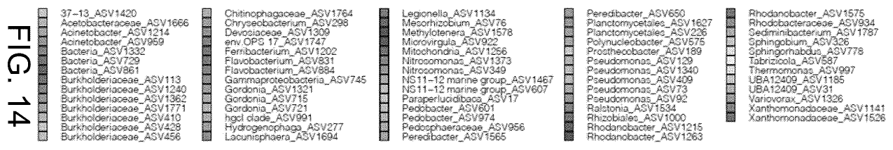

FIG. 15 is a chart illustrating a PCA analysis showing the effects of geosmin enrichment on microbial population diversity in BH aquaculture water.

FIG. 16 is a chart illustrating microbial population community diversity development, illustrated as colour-coded ASVs, within and on a mineral-based insulation, such as ROCKWOOL®, maintained in circulating BH aquaculture water amended with selected concentrations of geosmin. Microbial cultures were sampled from surfaces of ROCKWOOL® fibers and the circulating aquaculture water.

FIG. 17 is a colour-coding bar chart identifying the individual ASVs detected and illustrated in FIG. 16.

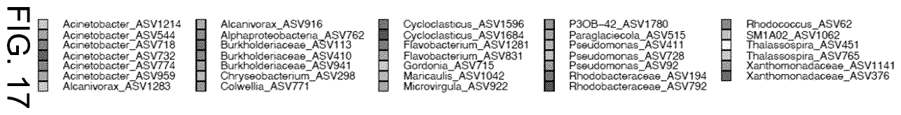

FIG. 18 is a chart showing microbial growth measured as changes in optical density (OD) at 600 nm, in geosmin-enriched BH aquaculture water.

FIG. 19 is a chart showing a time course of geosmin degradation by geosmin-enriched microbial populations in BH aquaculture water.

FIG. 20 is a chart illustrating a PCA analysis showing the diversity of ASVs from the geosmin-enriched microbial populations shown in FIG. 18.

FIG. 21 is a heatmap of the 10 most abundant ASV genera in the study with BH aquaculture water shown in FIGS. 18-20.

FIG. 22 is a chart showing microbial growth measured as changes in OD 600 nm, in low-geosmin-amended freshwater aquaculture water.

FIG. 23 is a chart showing microbial growth measured as changes in OD 600 nm, in higher-geosmin-amended freshwater aquaculture water.

FIG. 24 is a chart showing a time course of geosmin degradation by geosmin-enriched microbial populations in freshwater aquaculture water.

FIG. 25 is a chart illustrating a PCA analysis of 16S amplicon data showing the effects of low levels of geosmin enrichment on microbial population diversity in freshwater aquaculture water. For cultures with geosmin quantification the concentration is indicated by color. Samples with grey markers were not quantified. The relative geosmin concentration of the water sample TB_20200811 was set to 22, which was the concentration measured after adding geosmin to the 8n, TB4 and TB5 enrichments at the start of the experiment.

FIG. 26 is a chart illustrating a PCA analysis of 18S amplicon data showing the effects of higher levels of geosmin enrichment on eukaryotic population diversity in freshwater aquaculture water. For cultures with geosmin quantification the concentration is indicated by color. Samples with grey markers were not quantified. The relative geosmin concentration of the water sample TB_20200811 was set to 22, which was the concentration measured after adding geosmin to the 8n, TB4 and TB5 enrichments at the start of the experiment. The three cultures have very different eukaryotic communities.

FIG. 27 is a is a heatmap of the 20 most abundant ASV genera in the study with freshwater aquaculture water shown in FIGS. 23-26;

FIG. 28 is a heatmap of the 10 most abundant ASV genera in the study with BH aquaculture water shown in FIGS. 23-26.

FIG. 29 is a chart showing microbial growth measured as changes in OD 600 nm, in geosmin-amended freshwater aquaculture water.

FIG. 30 is a histogram that shows the percent removal of geosmin by various cultures.

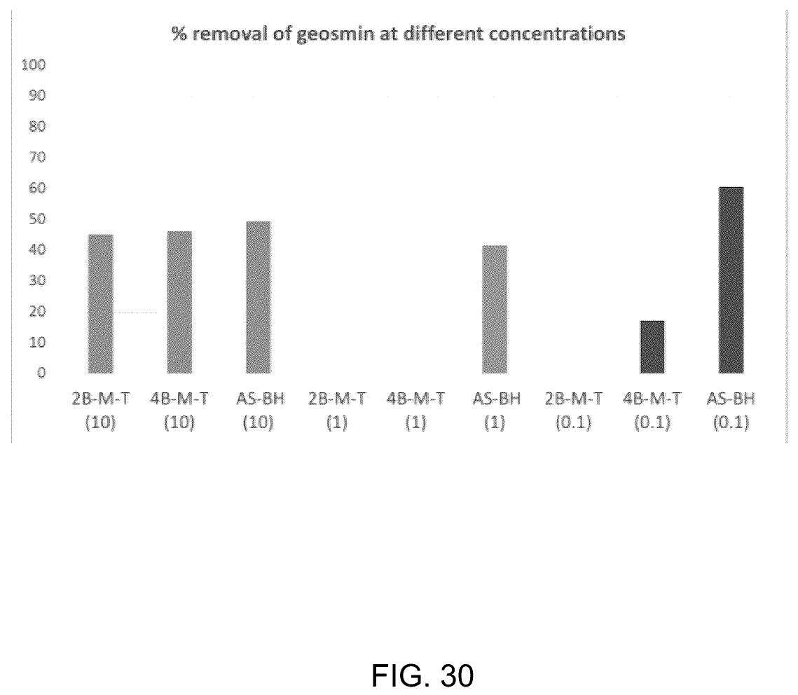

FIG. 31 is a histogram that shows the detected geosmin levels when measured in further samples treated with 100 μg/L of geosmin

FIG. 32 is a histogram that shows the percent removal of geosmin when measured in further samples treated with 10 μg/L of geosmin.

FIG. 33 is a histogram that shows the percent removal of geosmin when measured in further samples treated with 1 μg/L of geosmin.

FIG. 34 is a histogram that shows the detected geosmin levels when measured in further samples inoculated on a carrier and treated with 10 μg/L of geosmin.

FIG. 35 is a histogram that shows the detected geosmin levels when measured in samples inoculated on a carrier and treated with 10 μg/L of geosmin.

FIG. 36 is a further histogram that shows the detected geosmin levels when measured in samples inoculated on a carrier and treated with 1 μg/L of geosmin.

FIG. 37 is a chart illustrating microbial population community diversity development, illustrated as colour-coded ASVs, from multiple amplicon sequencing analyses on subsamples of the AS-BH consortium.

FIG. 38 is two histograms that show detected levels of (−)-geosmin when measured in further samples treated with 100 μg/L of geosmin, wherein FIG. 38A shows data from TO samples and FIG. 38B does not.

FIG. 39 is a histogram that shows detected levels of (−)-geosmin when measured in further samples treated with 10 μg/L of geosmin.

FIG. 40 is a histogram that shows detected levels of (−)-geosmin when measured in further samples treated with 1 μg/L of geosmin.

FIG. 41 is a histogram that shows detected levels of (−)-geosmin when measured in marine samples treated with 100 μg/L of geosmin.

FIG. 42 is a histogram that shows detected levels of (−)-geosmin when measured in marine samples treated with 10 μg/L of geosmin.

FIG. 43 is a histogram that shows detected levels of (−)-geosmin when measured in marine samples treated with 1 μg/L of geosmin.

FIG. 44 is a histogram that shows detected levels of (−)-geosmin when measured in samples treated with a metabolic substrate and 100 μg/L of geosmin.

FIG. 45 is a histogram that shows detected levels of (−)-geosmin when measured in samples treated with a metabolic substrate and 10 μg/L of geosmin.

### DETAILED DESCRIPTION

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

As used herein, the term “carrier” refers to a material that is suitable for combination and incubation with an enriched microbial consortium to thereby produce the microbiome aggregates.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the elements that it introduces.

As used herein, the terms “deploy”, “deploying” and “deployment” refer to introducing the compositions of the present disclosure into the water of a RAS so that the compositions can reduce or substantially remove one or more target, off-flavour compounds within the water of the RAS. In some embodiments of the present disclosure, the compositions may be deployed by being introduced directly into the water of the RAS. In some embodiments of the present disclosure, the compositions may adhere to a surface of a carrier that may be at least partially suspended in the water or the carrier may be in a fixed position with in the RAS.

As used herein, the term “desired amount” refers to an amount of a target compound that is desired to be reduced to in a given amount of water, for example RAS water, so that the negative impact of the presence of the target compound above the desired amount is reduced, substantially removed or entirely removed. For example, if the target compound is present in the water above the desired amount then certain mitigation efforts are required in order to reduce the negative impact, whereas the embodiments of the present disclosure may reduce the amount of the target compound below such desired amount so that such further mitigation efforts are less necessary or not necessary at all. In some embodiments of the present disclosure, the desired amount of an off-flavour compound is low enough that it cannot be detected within the water by an operator, either using specific sensors and instruments, or not. In some embodiments of the present disclosure, the desired amount of an off-flavour compound is low enough that it cannot be detected within a product being grown in the water by an operator, either using specific sensors and instruments, or not.

As used herein, the term “enrichment” refers to the culturing of a microbiome obtained from a sample of RAS in a selected medium supplemented with one or more of geosmin, 2-MIB, and cortisol, to select for and increase the abundance and biological activity of microbial species with the capacity to tolerate and degrade the selected one or more of geosmin, 2-MIB, and cortisol.

As used herein, the terms “inoculate”, “inoculating” and “inoculation” refer to combining and culturing an enriched geosmin-degrading microbiome and/or an enriched 2-MIB-degrading microbiome and/or an enriched cortisol-degrading microbiome with a selected carrier to produce agglomerated structures comprising mixtures of the enriched geosmin-degrading microbiome and/or enriched 2-MIB-degrading microbiome and/or enriched cortisol-degrading microbiome bound to the selected carrier via a biofilm. The agglomerated structures are referred to herein as “aggregates”.

As used herein, the term “metagenome” refers to the genomic materials of a microbiome from environmental samples, enrichment cultures, inoculated carriers, or other samples of microbiomes.

As used herein, the term “metagenomics” refers to the nucleic acid sequencing and analysis of the metagenome from environmental samples to produce taxonomic and functional gene profiles of the microbial community present in the samples.

As used herein, the term “microbial species” refers to all viruses, bacteria, archaea, fungi, and yeasts that are present in one or more samples collected from a recirculating aquaculture water system. “Microbial species” may also be referred to herein as “microbial populations”.

As used herein, the term “microbiome” refers to all of the microbial species present in a recirculating aquaculture system (RAS), and includes the microbial species present in aquaculture water, on the walls and floors of aquaculture tanks, in aquaculture water filter systems, in or on fish, and in or on fish feed. Additionally, the term “microbiome” encompasses the activity of the microbial species which results in the formation of specific ecological niches. A microbiome will form a dynamic and interactive micro-ecosystem that is prone to change in time and scale, and which, may be integrated in into selected macro-ecosystems

As used herein, the terms “recirculating aquaculture system”, “recirculating aquaculture water system”, and the acronym “RAS” all of which may be used interchangeably and refer to systems in which one or more types of products, such as marine and freshwater fish, crustaceans and the like, can be grown in a controlled environment. Recirculating aquaculture systems typically include one or more tanks for containing and growing the products, a pumping system and a waste removal/treatment system. Typical pumping systems include pumps for circulating (and recirculating) water through the one or more tanks, the waste removal system and the conduits that fluidly connect them. The pumping system may also be configured to replenish oxygen within the circulating water. The waste removal systems are typically configured to reduce the levels of waste within the circulating water. Such wastes include chemical waste, such as nitrogen-containing compounds (for example, ammonia), carbon dioxide and solid waste.

As used herein, the term “16S analysis” means use of 16S rRNA gene sequencing of microbial populations present in a sample, for identification of and taxonomic grouping of the bacterial species present in the sample.

The embodiments according to the present disclosure generally relate to compositions for deployment into a RAS environment, wherein such compositions are configured to reduce, substantially remove or completely remove one or more target compounds within the circulating water of the RAS. The compositions comprise an enriched microbiome that is adhered to a surface of a carrier by a biofilm. In some embodiments of the present disclosure, the carrier may induce the enriched microbiome to produce the biofilm, or produce more of the biofilm, to facilitate adherence of the enriched microbiome. In some embodiments of the present disclosure, the carrier is configured to selectively sequester a target compound from the water of a RAS environment. This sequestering can be due to the carrier adsorbing or absorbing the target compound onto its surface so as to bring the target compound into functional proximity of the enriched microbiome.

In some embodiments of the present disclosure, the carrier may be a particle that is suspendible within the water of the RAS environment. In some embodiments of the present disclosure, the carrier is a fixed, constructed or installed component of the RAS that includes one or more of a filter, a tank liner, a conduit liner or combinations thereof. For example, the carrier may define a surface of a fixable component of the RAS and/or the carrier may define a surface of a filter, a tank liner, a conduit liner or combinations thereof. In some embodiments of the present disclosure, the carrier may be deployed into the RAS water by being placed in operable communication with the water of the RAS. For example, the carrier may be placed directly in the RAS water loop or the carrier may be placed in a side stream or slip stream loop of the RAS system. For example, the carrier may be in a parallel fluidic circuit and/or a series fluidic circuit as part of the RAS.

In some embodiments of the present disclosure, the carrier may comprise a substance with a wax-like consistency. Such waxy carriers can take many forms from a bead that can be suspended within the water of a RAS.

Additionally or alternatively, the waxy carriers may be formed into any shape suitable for incorporating into, on to or to form at least part of a surface of a fixed component of the RAS, such as a tank, conduit and/or filter. In these examples, the waxy carrier may be in direct contact with the water of the RAS. Non-limiting examples of waxy carriers include: paraffin or camphor and other similar waxy substances.

In other embodiments of the present disclosure, the carrier may comprise fresh and or saltwater metazoan, such as live copepods, copepod eggs, copepod carcass, zooplankton, krill, microalgae, macroalgae and other suitable members of the applicable metazoan.

In some embodiments of the present disclosure, the carrier may comprise one or more: of a hydrogel; a biopolymer such as alginate; a suitable moving bed reactor substrate, such as plastic bioreactor beads of any suitable morphology or made by any method of manufacture; a mineral-based insulation for example, ROCKWOOL® (ROCKWOOL is a registered trademark of Rockwool International A/S, Hedehusene, Denmark) and other mineral-based insulation products; fish feed, clay and particles of clay.

Some embodiments according to the present disclosure are related to methods for preparing the compositions disclosed herein.

Some embodiments according to the present disclosure relate to systems for rapid delivery and deployment of the compositions disclosed herein, into the water of a RAS. Some embodiments of the present disclosure relate to the composition forming part of the RAS environment, for example as forming a surface of a fixable component of the RAS.

According to one example embodiment, a method for preparing a composition configured for deployment into a RAS environment, may comprise the steps of:
1. collecting a water sample from a source of water that comprises a microbiome and that has been exposed to one or more target compounds, for example, at an aquaculture production facility or otherwise;
2. enriching the microbiome present in the water sample by culturing in a selected liquid medium supplemented with the one or more target compounds for a predetermined period time, for example, of at least ½ day, 1 day, 2 days, 3 days, 4 days, 5 days, 6 days, 7 days or more, for example 10 days or more, 14 days or more, 18 days or more, 21 days or more, or longer, to thereby produce an enriched microbiome;
3. combining the enriched microbiome with a selected carrier or an additive, by the presence of a biofilm generated by the enriched microbiome or not. The enriched microbiome and carrier are then incubated for a desired time period (for example, more or less than 18 hours) while gently commingling the enriched microbiome and carrier, for example at a rate selected from a range of about 0.5 RPM to about 30 RPM, to produce the composition. Alternatively, the enriched microbiome and the additive may be mixed together.

In some embodiments of the present disclosure, the enriched microbiome may be supplemented with an additive to facilitate at least partially suspending the enriched microbiome within the RAS water. The additive may be any type of chemical compound that facilitates suspension of the enriched microbiome within the RAS water. Optionally, a biofilm may enhance or facilitate integration of the enriched microbiome and the additive so that the additive may impart physicochemical properties upon the enriched microbiome, for example, the mixture of the enriched microbiome and agent may become at least partially suspended within the RAS water. In contrast, the enriched microbiome alone (without additive) may settle to the bottom of the tanks or conduits within the RAS, thereby reducing the effect of the enriched microbiome upon the RAS water.

In some embodiments of the present disclosure, the agent may act as a growth substrate, or a co-substrate for cometabolic enrichment, to facilitate enriching the microbiome communities.

The composition may comprise agglomerated microbial components of the enriched microbiome and carrier particles that are loosely bound together by biofilms formed and secreted by the microbial components of the enriched microbiome. The agglomerated structures may also be referred to herein as “aggregates”. In other words, the present composition may comprise aggregates that include microbial components of the enriched microbiome attached to carrier particles by biofilms secreted by the microbial components. In some embodiments of the present disclosure, the carrier particles may allow the enriched microbiome to be fixed to a surface of a carrier that can be deployed within the RAS. In some embodiments of the present disclosure, the carrier particles may allow the enriched microbiome to be, at least partially, suspended within the water of the RAS so that the compound moves through the fluid circuit of the RAS with the circulating water. In some embodiments of the present disclosure, the composition may comprise agglomerated microbial components and a biofilm and both are configured to be secured to the surface of a carrier. In some examples of the present disclosure, the carrier may be a fixable component of the RAS infrastructure, such as a liner of a tank, fluid conduit, a filter of the waste management system, another component of the waste management system or combinations thereof.

According to some embodiments of the present disclosure, a suitable medium for culturing and enriching a microbiome present in a RAS sample may be Buschnell Haas mineral broth and other types of media nutrient broth, such as: Väätänen Nine-Salt Solution (VNSS), marine broth, Luria-Bertani marine broth, 2% NaCl Mueller-Hinton broth, Zobella marine broth, combinations thereof and the like.

According to some embodiments of the present disclosure, a suitable target, off-flavour compound for culturing and enriching a microbiome present in a RAS sample may be a terpene or terpenoid for culturing and enriching a microbiome present in a RAS water sample. Specific examples of a suitable terpene or terpenoid may be one of or both of geosmin and 2-MIB.

According to some embodiments of the present disclosure, a suitable target compound for culturing and enriching a microbiome present in a RAS sample may be a steroid. A specific example of a suitable corticosteroid may be a corticosteroid such as cortisol.

According to some embodiments of the present disclosure, a suitable mixture of target compounds may be a mixture of or more of a terpene, a terpenoid, an amine-based compound, a haloanisole, a steroid such as a corticosteroid like cortisol, or combinations thereof.

According to some embodiments of the present disclosure, the enriching step for culturing terpene or terpenoid-degrading and/or steroid-degrading microbial populations present in the microbiome of a collected RAS sample may be done at a culturing temperature selected from a range of about 1° C. to about 30° C. A particularly suitable temperature range for culturing terpene or terpenoid-degrading and steroid-degrading microbial populations present in the microbiome of a collected sample may be from a range of about 5° C. to about 25° C.

According to some embodiments of the present disclosure, enriched terpene or terpenoid-degrading microbiomes and/or steroid-degrading microbiomes produced according to above method steps may comprise a plurality of microorganisms such as Rhodopseudomonas, Acanthopleuribacter spp., Acinetobactor spp., Alcanivorax spp., Alteromonadaceae spp., Cycloclasticus spp., Flavobacterium spp., Hyphomonas spp., Marinobacter spp., Pseudomonas spp., Rhodobacteraceae spp., Sphingopyxis spp., Sphingobium spp., Xanthobacter spp., among others. Some of the enriched organisms may be terpene, terpenoid and/or steroid degraders, while others may be involved in biofilm formation and dead-biomass recycling.

The methods may comprise a step of maintaining an enriched terpene or terpenoid-degrading and/or steroid-degrading microbiome by one of a continuous culture process or a batch culture process. If a continuous culture process is selected for maintaining an enriched terpene or terpenoid-degrading and/or steroid-degrading microbiome, then the selected nutrient medium and the selected terpene or terpenoid-degrading and/or steroid-degrading product may be supplied to the enrichment culture vessel at selected constant rates while enriched microbiome is removed from the enrichment culture vessel at a rate equivalent to the input rates. If a batch culture is selected for maintaining an enriched terpene or terpenoid-degrading and/or steroid-degrading microbiome, then at a selected time wherein the enriched microbiome is in a steady state, the batch culture be separated into two or more portions wherein one of the portions is transferred to a fresh batch culture vessel containing therein the selected nutrient medium and the selected terpene or terpenoid-degrading microbiome and/or steroid-degrading microbiome for continued enrichment and maintenance of the terpene or terpenoid-degrading and/or steroid-degrading microbiome.

The next step may involve combining the volumes of enriched terpene or terpenoid-degrading and/or steroid-degrading microbiomes with one or more selected carriers, and then gently mixing the combination at a rate from about 0.5 RPM to about 30 RPM for a selected time period to produce the desired compositions that are suitable for delivery into a RAS. Suitable time periods may be 2 h, 4 h, 8 h, 12 h, 16 h, 20 h, 24 h, and therebetween. It is optional to select longer time periods if so desired, for example 36 h, 48 h, 60 h, or longer.

It is an optional opportunity during the maintenance of an enriched terpene or terpenoid-degrading and/or steroid-degrading microbiome culture, to combine the portions of enriched microbiome cultures harvested and removed during the maintenance operations, with a selected carrier and then gently mixing the combination at a rate from about 0.5 RPM to about 30 RPM for 24 h or longer to produce compositions that are suitable for routine regular delivery into selected RAS.

Without being bound by any particular theory, some embodiments of the present disclosure relate to compositions, systems and methods that take advantage of enriching microbiomes that comprise one or more constitutive members that are obligate degraders of one or more target compounds. In some embodiments of the present disclosure, the one or more steps of enriching the microbiome include one or more steps of limiting the carbon available for members of the microbiome to utilize for metabolic processes. Without being bound by any particular theory, providing the target compound as the single carbon source may select for increased amounts, presence and/or metabolic activity (on a relative level compared to other members of the microbiome community) of the desired obligate degraders, which may result in increased degrading of the one or more target compounds because the desired obligated degraders are using the one or more target compounds as a source of carbon. In this fashion, the enriched microbiome may be enriched for desired obligate degraders.

Some embodiments of the present disclosure, either in addition to or separate from step of enriching on the target compound, comprise one or more steps that rely on one or more cometabolic enrichment processes. During steps of cometabolic enrichment, a co-substrate is used as a source of selection pressure, instead of the target compound. Cometabolic enrichment also may or may not result in obligate degraders using the one or more target compounds as a source of carbon. Cometabolic enrichment has the advantage that degradation of the target compound to zero, low or trace concentrations is possible, since the community is not dependent on the contaminant for carbon or energy, As but one non-limiting example, employing one or more steps of cometabolic enrichment may result in one or more aspects of a microbiome member's metabolic machinery, such as one or more metabolic proteins (including enzymes), being activated (for example: increased expression of the genes whose transcription products comprise the one or more metabolic proteins, increased post-translational modifications of such one or more metabolic proteins, increased metabolic activity of such one or more metabolic proteins and combinations thereof) to degrade the co-substrate (which is not directly related to metabolism of the target compound) that is added (e.g. maltose, ammonium). As used herein, the terms co-substrate and substrate are used to refer to a compound that can be supplied to an enriched microbiome, where such compound can be metabolised by the enriched microbiome where such metabolic activity enhances the enriched microbiome's degradation, directly or indirectly, of the target compound. As a further example, a microbiome may be cometabolically enriched to increase the presence of constitutive members that have an enzyme suitable for metabolizing a compound that is known to be unrelated to metabolic break down of the target compound. As such, when this enriched microbiome is loaded onto a suitable carrier and deployed into a RAS, the water of the RAS may be treated with the substrate. This treatment may cause the enriched microbiome to metabolize (degrade, breakdown, or otherwise decrease the circulating levels of) the unrelated compound and any target compound that may also be present in the water that is proximal to the carrier.

Some embodiments of the present disclosure may employ steps of enriching that include selecting based on target-compound enrichment and one or more cometabolic enrichment processes (for example, by employing one or more unrelated metabolic co-substrates).

### EXAMPLES

### Example 1: Proof of Concept Study

For the first set of geosmin degradation experiments, water was collected from two different aquaculture facilities in northern Europe, a first facility is referred to herein as “AS” and the second facility is referred to herein as “BH”. Dense growing enrichment cultures were established in Buschnell-Haas nutrient media supplemented with geosmin dissolved in ethanol as the only added carbon source.

Both water sources, especially AS, resulted in the accumulation of red biofilm clumps. These biofilm clumps are also visible in the water sources at lower quantities.

Geosmin was filter sterilized using a 0.22 um syringe filter. For the negative control, the water was filtered through a 0.22 μm filter.

The geosmin used was dissolved in 100% EtOH and appropriate controls were used where EtOH was supplied as a carbon source. Both water sources also had organic debris, and therefore appropriate controls for nutrients only were included.

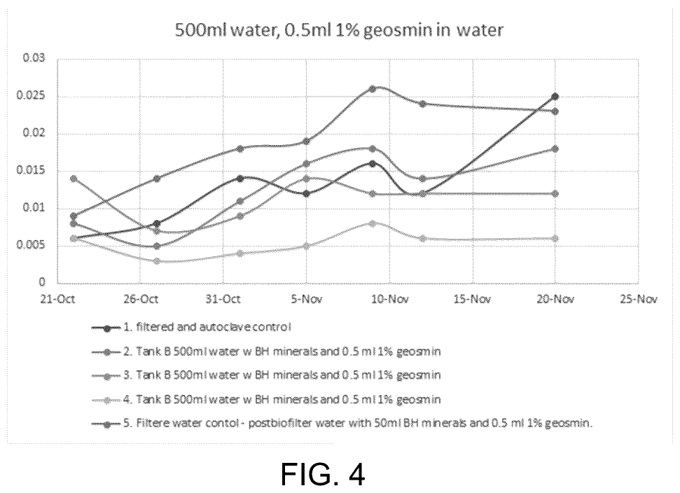

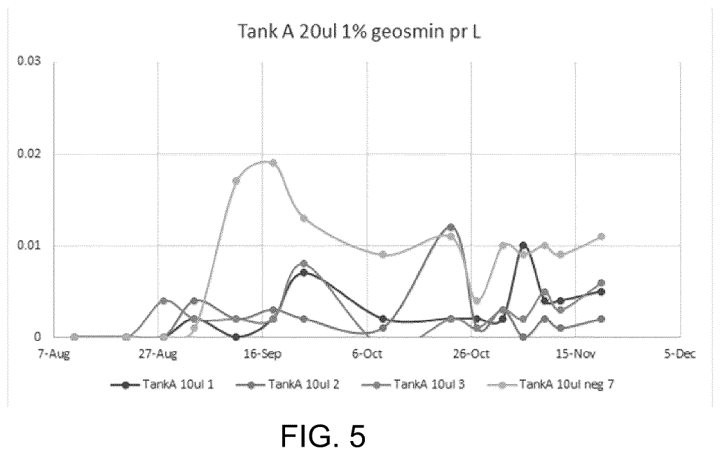

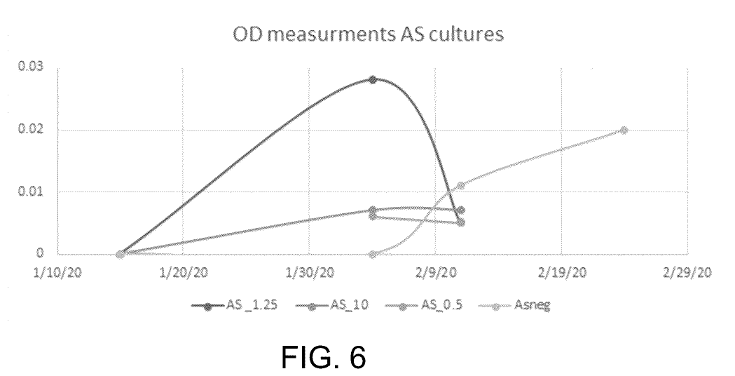

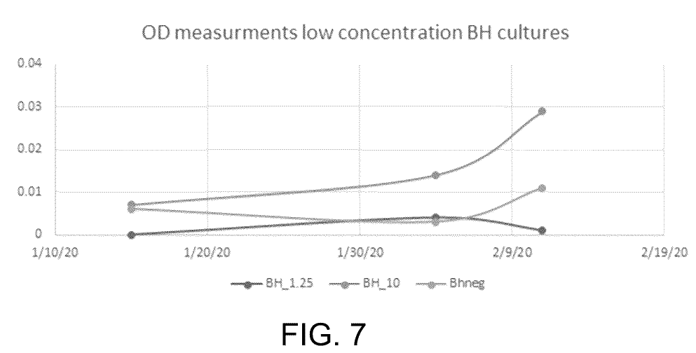

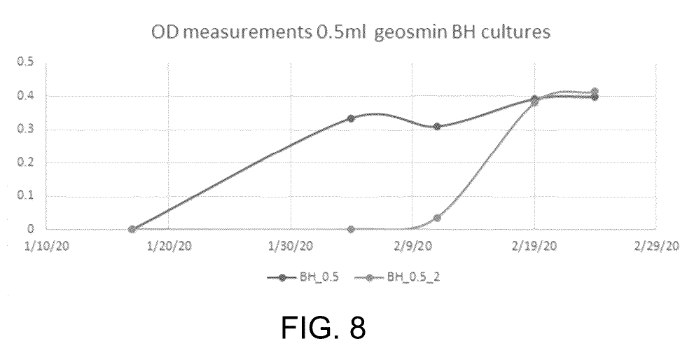

Optical density (OD) measurements were taken regularly. Since biofilm clumps tended to form in all of the low amount bottles, these measurements do not fully capture the growth of the enrichments. However, the OD measurements do reflect the better growth in the BH enrichments (see FIGS. 4-8, where OD 600 is shown on the Y axis and time points are shown on the x axis).

The samples were then analyzed by headspace solid-phase microextraction-gas chromatography-tandem mass spectrometry (herein referred to as HSSPME-GC-MS/MS). One quality control standard (QC), 1 method blank (MB), and 2 duplicate samples (+Dup) were included. The samples were refrigerated about at 7° C. before aliquoting for analysis. Aliquots of about 2 mL of each sample were diluted with about 8 mL of 18 MΩ water from a pure water dispenser (Elga LabWater via VWR International, Mississauga, Ontario). Samples were then sealed in a 20-mL screw-cap glass headspace vials with about 3 g NaCl after the addition of about 1 μL of 230 mg/L benzaldehyde-d5 and dodecyl alcohol-d25 internal standards. Dodecyl alcohol-d25 was added as a supplementary QC step to track the impact of samples on organic internal standards; quantification was all done using benzaldehyde-d5.

The headspace vials were then incubated at about 60° C. for about 10 minutes, followed by extraction without agitation using a polydimethylsiloxane (PDMS) SPME fiber with a 100 um phase thickness for 60 min. Samples were then desorbed for 5 min into the injector of the gas chromatograph at about 250° C.

Separation was performed on a BRUKER® 456 gas chromatograph with a triple quadrupole (QqQ) BRUKER® SCION® mass spectrometer (BRUKER and SCION are registered trademarks of Scion Instruments Corp, Austin, TX, USA) operated in electron impact ionization mode for detection. The GC column was a 30 m×0.25 mm, 0.25 um film thickness DB-5 column (Agilent Technologies, Mississauga, ON). The mass spectrometer was operated in MRM mode using m/z transitions of 112.0→97.1, 112.0→83.1, and 112.0→69.1 for geosmin. The benzaldehyde-d5 internal standard was also analyzed in MRM mode, monitoring m/z transitions 111.0→82.1, 110.0→82.1, and 77.0→54.1. Dodecyl alcohol-d25 was analyzed by monitoring 62.3, 78.3, and 94.4 m/z ions in Q1 and Q3 transmission mode. QC mix standards also included acetophenone-d5 and n-hexane. Acetophenone was analyzed using the same MRM transitions as benzaldehyde-d5, while hexane was analyzed by monitoring the ion 57.0 m/z in Q1 with Q3 run in transmission mode.

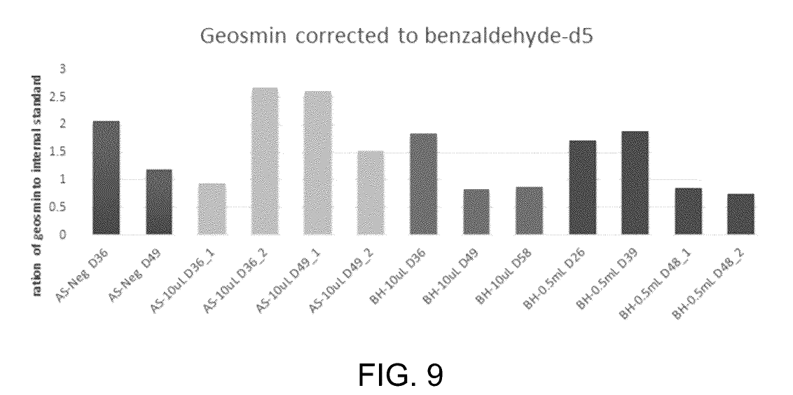

HSSPME-GC-MS/MS geosmin quantification indicates that the BH community may degrade geosmin (as shown in FIG. 9). To conduct these studies, geosmin in the two sample enrichments (AS and BH) was normalized to a benzaldehyde-d5 internal standard. On the x axis, “Dxx” indicates days since start of experiment. All samples were diluted to an initial concentration of 20 μg/L. Note that two measurements were done on the sample from AS-10 ul D36, AS-10 ul D49 and BH-0.5 ml D48.

The loss of geosmin in the negative control bottle may be due to degradation of geosmin by bacteria that could pass through the filter. Growth was observed in the AS negative control at the point the sample with low geosmin was taken, and the bacterium (Alcanivorax) in this sample might be responsible for the degradation, discussed further below.

For relevant examples described here, DNA was isolated from each of the stored samples using the PowerLyse Power Soil kit from Qiagen (Toronto, CA) following the instructions in the kit's manual. DNA concentrations in the samples were determined with a QUBIT® fluorometer (QUBIT is a registered trademark of Qubit LLC, Plano, TX, USA). The DNA samples were sent to Microbiome Insights (Vancouver, BC, CA) for 16S rRNA amplicon sequencing. Primers used for 16S rRNA amplification were (i) the 515F primer “GTGCCAGCMGCCGCGGTAA”, and (ii) the 806R primer “GGACTACHVGGGTWTCTAAT”. 16S rRNA amplicon data were processed to amplicon sequence variants using the QIIME2 platform (Bolyen et al., 2019, Reproducible, interactive, scalable and extensible microbiome data science using QIIME 2. Nature Biotechnology 37:852-857) using DADA2-plugin (Callahan et al., 2016, High-resolution sample inference from Illumina amplicon data. Nature Methods 13:581-583) and visualized using the phyloseq in R package for microbiome census data (McMurdie and Holmes, 2013, phyloseq: An R Package for Reproducible Interactive Analysis and Graphics of Microbiome Census Data. PLOS ONE 8: e61217).

Table 1 below identifies the various samples and conditions from which DNA was isolated for 16S rRNA analysis.

TABLE 1
Summary of 16S rRNA analysis

| | | Amount 1% | | Days of |
|---|---|---|---|---|
| Sample id | Sample type | geosmin^{a} | | incubation |
| ASw0601 | water | NA | | NA |
| BHw0601 | water | NA | | NA |
| ASf3 | water | NA | | NA |
| ASEtOH212 | etoh control | NA | | NA |
| BHEtOH212 | etoh control | NA | | NA |
| ASnut212 | nutrient control | NA | | NA |
| Bhnut_231 | nutrient control | NA | | NA |
| Asneg_242 | enrichment | 10 | ul | 50 |
| AS1_25_291 | enrichment | 1.25 | ul | 24 |
| AS1_25_112 | enrichment | 1.25 | ul | 37 |
| AS10_291 | enrichment | 10 | ul | 24 |
| AS10_242 | enrichment | 10 | ul | 50 |
| AS1mg_291 | enrichment | 0.5 | ml | 12 |
| AS1mg_242 | enrichment | 0.5 | ml | 38 |
| Bhneg_231 | enrichment | 10 | ul | 17 |
| BHneg_42 | enrichment | 10 | ul | 29 |
| BH1_25_231 | enrichment | 1.25 | ul | 17 |
| BH1_25_42 | enrichment | 1.25 | ul | 29 |
| BH1_25_242 | enrichment | 1.25 | ul | 50 |
| BH10_231 | enrichment | 10 | ul | 17 |
| BH10_42 | enrichment | 10 | ul | 29 |
| BH10_242 | enrichment | 10 | ul | 50 |
| BH1mg_231 | enrichment | 0.5 | ml | 6 |
| BH1mg_42 | enrichment | 0.5 | ml | 18 |
| BH1mg_242 | enrichment | 0.5 | ml | 38 |

n.b. 1 geosmin in ethanol per 500 ml of culture.

### AS Samples

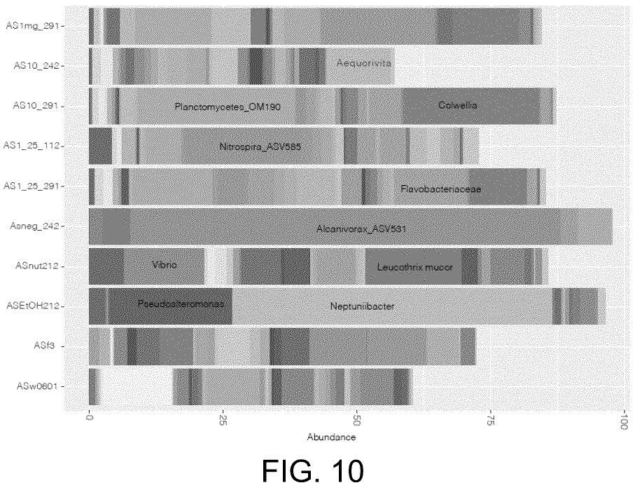

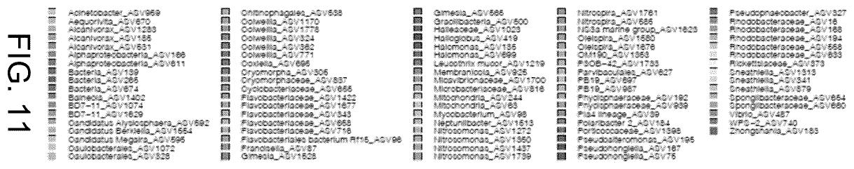

FIG. 10 and FIG. 11 provide taxonomic distribution data from the AS samples for the amplicon sequence variants (ASVs) identified therein. These ASVs are present in at least greater than 1% of the AS samples assessed. The two first samples (ASw0601 and ASf3) represent the microbial community in the water before the start of the experiment. ASw0601 and ASf3 were filtered.

The ASneg242 was the negative control bottle used after growth was seen in this sample on February 24. The dominant ASV in this sample is Alcanivorax ASV531 (>50,000 reads), which was not observed at high levels in any other sample. This ASV was also observed in the 1 mg bottle at the latest time point.

The control with EtOH only, was very dense and contained mainly Neptuniibacter and Pseudoalteromonas. The control with nutrients only, contained Leucothrix mucor, Vibrio and Pseudophaeobacter. None of these species were observed at high levels in the geosmin enrichments, with the exception of Neptuniibacter in the AS1 mg enrichment from 29/1, suggesting that the organisms detected in the geosmin enrichments metabolize the geosmin.

All the AS geosmin enrichments contain the same set of core organisms not observed at high levels in the controls; Planctomycetes_OM190 (ASV1353), Flavobacteriaceae (ASV1677), Nitrospira (ASV585) and Colwellia (ASV362). These organisms may constitute a geosmin-degrading consortium with one or more of these organisms metabolizing the geosmin.

The Planctomycetes_OM190 is an uncultured Planctomycetes lineage common in marine environments and have also been observed in aquaculture facilities by others. These bacteria are biofilm formers and are common in marine snow, and could be responsible for the biofilm ‘clumps’ observed in the bottles. Many Planctomycetes conduct “anammox” metabolism, a process in which ammonia is oxidized by nitrate to nitrogen gas, yielding energy.

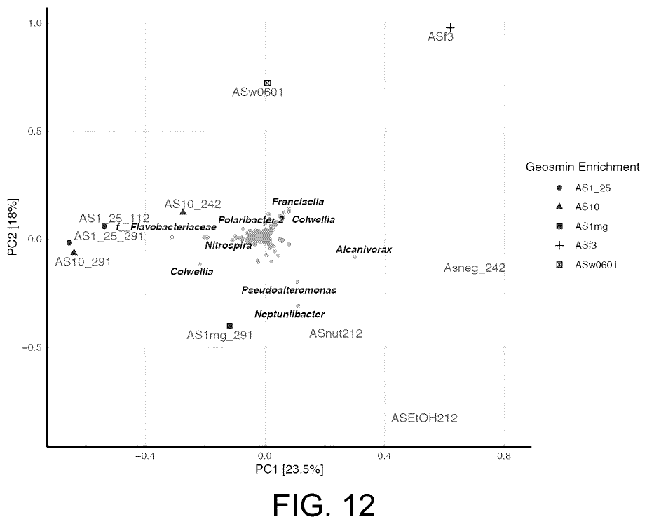

A PCA plot with the 10 top species added is shown in FIG. 12. The ordination shows the geosmin enrichments clustering together. The PCA plot of AS source water (ASw0601, ASf3), geosmin enrichments (AS1_25, AS10, AS1 mg) and controls (ASnut212, ASneg_242, ASEtOH212) is shown in FIG. 12. Prior to the analysis, ASV's that were not present in more than 0.1% relative abundance in any sample have been removed. The data has been transformed initially by applying the hellinger transformation (per Legendre & Gallagher, 2001). The relative contribution (eigenvalue) of each axis to the total inertia in the data is indicated in percent at the axis titles.

### BH Samples

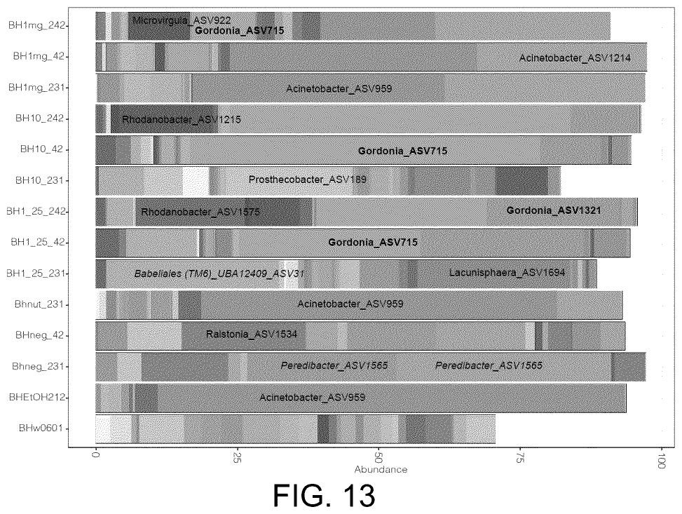

The data in FIG. 13 is of ASVs present in greater than 1% of the BH samples. The controls grown on EtOH and nutrients only, contain high abundances of Acinetobacter ASV959, suggesting that this organism can grow on EtOH and/or the biomass available in the BH water. This ASV is also the most abundant in the enrichments that had 1 ml/L 1% geosmin in EtOH added. Acinetobacter has been reported to degrade geosmin, however, the high abundance in the control samples makes it difficult to determine with certainty if the Acinetobacter observed in our samples degrades geosmin.

The enrichments with lower amounts of geosmin (1.25 ul and 10 ul 1% geosmin per 500 ml culture) contain high relative abundances of two Gordonia ASVs (ASV715 and ASV1321), which are the most likely geosmin degrading organism in these cultures. Note that ASV715 also increases in abundance in the enrichment with 1 ml/L (FIG. 13). Most Gordonia species were isolated due to their known abilities to degrade xenobiotics, environmental pollutants, or otherwise slowly biodegradable natural polymers as well as to transform or synthesize possibly useful compounds. The Rhodanobacter ASV1215 is a common soil bacterium and the higher abundance of the bacterium in both the 1.25 and 10 ul could suggest it is also involved in geosmin metabolism.

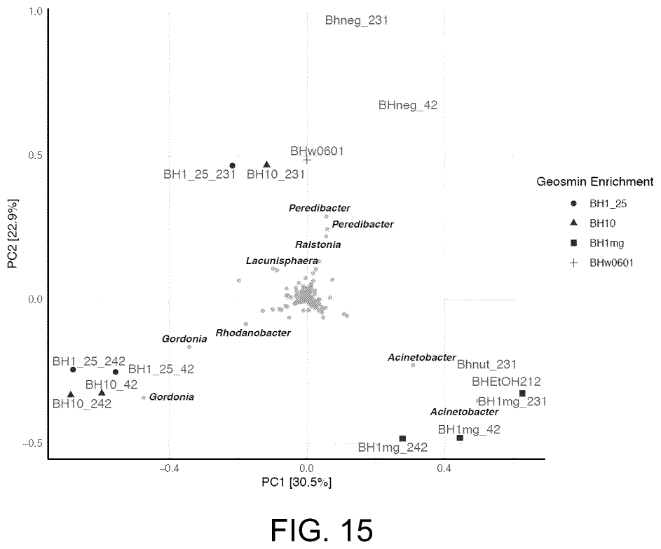

The PCA plot in FIG. 15 supports the above conclusions. The 1 ml/L 1% geosmin culture clusters with the control bottles along the first axis, due to the high abundance of Acinetobacter. The first time points of the 1.25 and 10 ul enrichments (23/1) are similar to the original water sample and then get enriched for the Gordonia ASVs (ASV715 and ASV1321) at the two later time points (4/2 and 25/2).

Addition of geosmin changed the community in both AS and BH enrichments suggesting that the microorganisms are either tolerate the geosmin better than other members of the initial community or that they are metabolizing (degrading) it. The BH enrichments on low concentrations were particularly helpful in identifying potential degraders and based on the results from these samples, a likely geosmin degrader belonging to the genus Gordonia was identified. An Acinetobacter might also be involved in degradation, however, this organism also grows on ethanol alone.

### Example 2: Loading on Carrier

One of the BH enrichments on 1 ml 1% geosmin/L cultures were used to load onto a carrier (as referred to as inoculating the carrier). In this example, the carrier was a ROCKWOOL® material.

ROCKWOOL® insulation sheeting (ROXUL® COMFORTBOARD® insulated sheathing; ROXUL and COMFORTBOARD are registered trademarks of Rockwool International A/S, Hedehusene, Denmark), in approximately 1 cm thick pieces, was added to roller tubes with an ‘conditioning’ solution and autoclaved. Conditioning solutions used were: Distilled water, Buschnell Haas nutrients, Tris buffer, or TE buffer.

The tubes were incubated over night before inoculation. ROCKWOOL® from 1 tube from one of each of the distilled water, Tris and BH-nutrient treated ROCKWOOL® was transferred to 6 new roller tubes and 5 ml of geosmin enrichment and 5 ml of filtered BH water were added. After overnight incubation on a roller disk, a biofilm developed on all ROCKWOOL® pieces where enrichment was added.

DNA was isolated from the inoculated ROCKWOOL®, and the taxonomic distribution of the communities was assessed by 16S rRNA amplicon sequencing to see if the enriched microbial community colonized and made up the biofilm on the ROCKWOOL®.

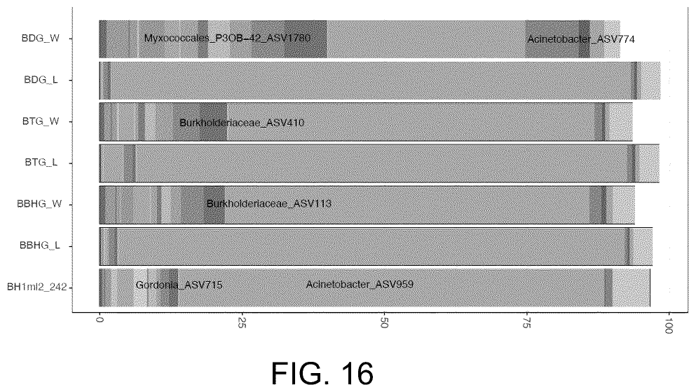

FIG. 16 shows a bar chart of ASVs present at >1% in the ROCKWOOL® samples. BH1 ml2_242 was the geosmin enrichment used. Samples with ‘_L’ added corresponds to the liquid fraction while ‘W’ samples are from the ROCKWOOL® fibers. Here “B” indicates a BH sample source and “BH” indicate that the ROCKWOOL® was treated with Buschnell Haas nutrients, “T” indicates pre-treatment with Tris and “D” pre-treatment with distilled water. For example, a BH sample source used to load onto the ROCKWOOL® and conditioned with Busnell Haas nutrients is shown as “BBH” whereas “BT” indicates a BH samples source used to load onto ROCKWOOL® and conditioned with Tris.

The taxonomic distribution in FIG. 16 shows that all the major organisms in the enrichment are also found in the ROCKWOOL®. Moreover, the diversity increased in the ROCKWOOL®, suggesting that some of the ASVs with lower abundance in the enrichment are better adapted to growth on surfaces than in liquid culture, for example the Burkholderiaceae ASVs.

### Example 3: Further Enrichment Studies

A second set of enrichments using BH water was performed. Triplicate 500 ml cultures were prepared with 1 ml 1% geosmin added per L. The geosmin was dissolved in EtOH and, without being bound by any particular theory, the communities will likely consume the ethanol before degrading the geosmin directly, or removing the geosmin as part of a cometabolic process. 10 ml samples were collected throughout the experiment for geosmin quantification. 2 ml samples were collected for DNA at the same time and OD was measured.

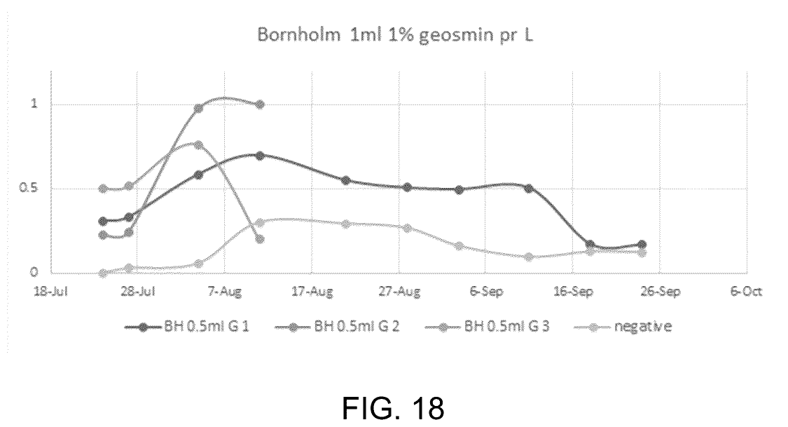

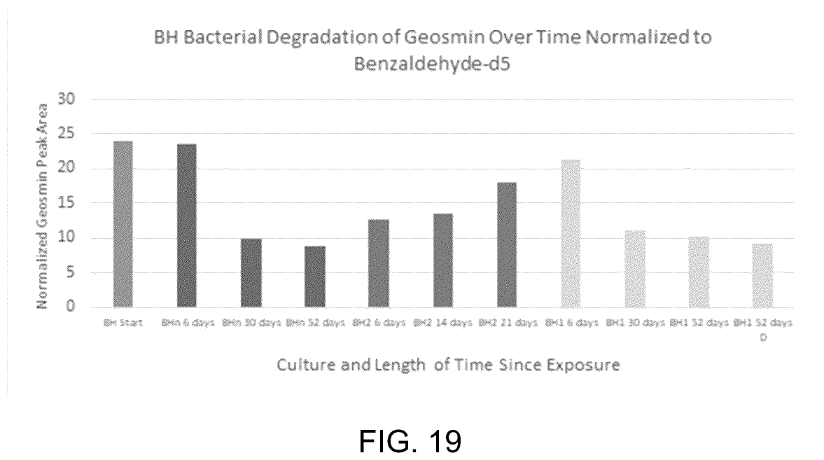

FIG. 18 shows the OD data and FIG. 19 shows the geosmin levels detected in the various samples. The geosmin concentrations observed in FIG. 19 suggests good degradation in the BHn and BH1. BH2 also seemed to have been degrading initially, but since there was an increase between day 14 and 21, this community might also be producing geosmin.

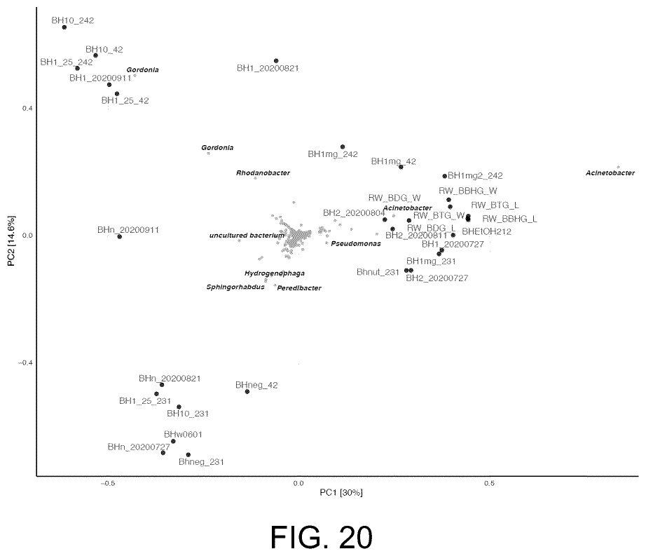

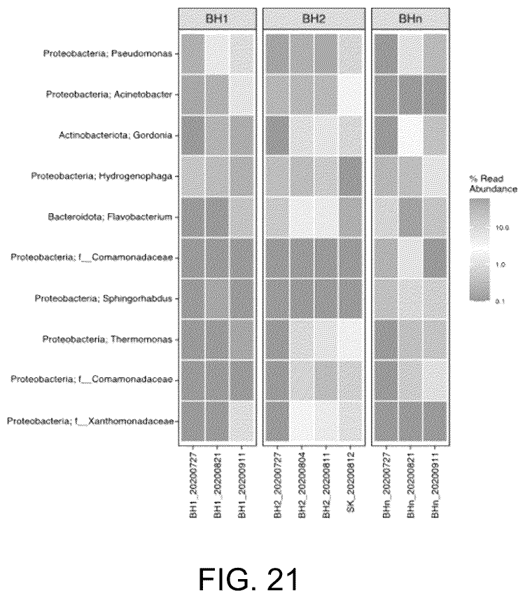

FIG. 20 shows the PCA plot of these samples. The filtered water (“negative controls”) clustered together, demonstrating that the same filterable-communities may be enriched independently. Moreover, the later time points of the BH1culture also contained a good degrading community, clusters with the BH10 ul culture from the first set of enrichments. Both enrichments are characterized by higher abundance of Gordonia, which data from Example 1 suggested was a geosmin degrader. A heatmap of the ten most abundant ASVs in the latest set of experiments is shown in FIG. 21.

### Example 4: Further Carrier Loading/Inoculating

In this Example 4, it was investigated whether feed used in RAS could be used as a carrier. Culture BH2 were used to inoculate feed (commercially available from Skretting SK20200812). Note, for this example filtered water from Example 3 water was used for the inoculation. The feed was inoculated in a 1000 ml bottle containing 0.12 g of ground up feed, 200 ml of the enrichment culture and 800 ml of filtered water taken from Example 3. The bottle was incubated overnight on a plankton wheel.

Sequence analysis of the inoculated feed shows that all the major genera from the BH2 culture is found on the feed demonstrating that feed can be loaded with an enriched microbiome according to embodiments of the present disclosure. It was observed that there was a higher relative abundance of Flavobacteium in the inoculated feed. These bacteria are common in soil and freshwater. They degrade carbohydrates, and are likely growing on the feed.

### Example 5: Further Water Samples

Water samples were received from several tanks at an aquaculture facility in southem Alberta, referred to herein as Tank A (TA) and Tank B (TB).

Triplicates of low (20 ul 1% geosmin/L) and high (1 m 1% geosmin/L) concentration cultures were set up and sampled for DNA, geosmin quantification and OD measurements on a weekly basis. Growth was observed in negative controls after an initial lag phase. These are likely small filterable cells as seen for the BH enrichments described above. The growth in the negative controls looks very different from the enrichment cultures. The enrichment cultures have large amounts of white and brown-red biofilm while the negative has less biofilm and is white and pink in color.

For these enrichments, the geosmin was dissolved in EtOH, and the communities will probably use the ethanol before the geosmin or simultaneously if co-metabolic.

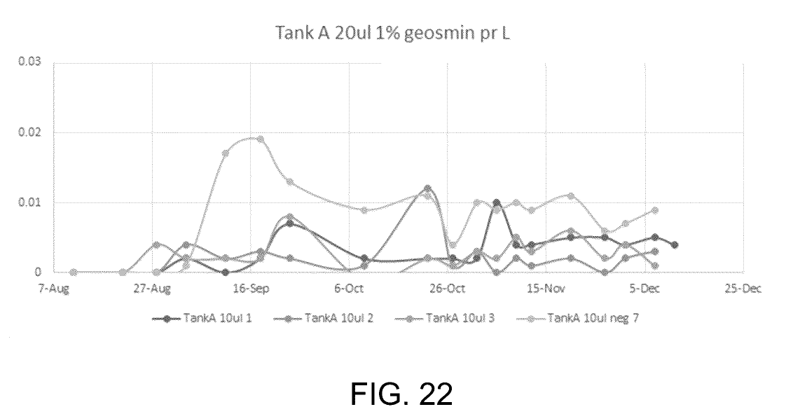

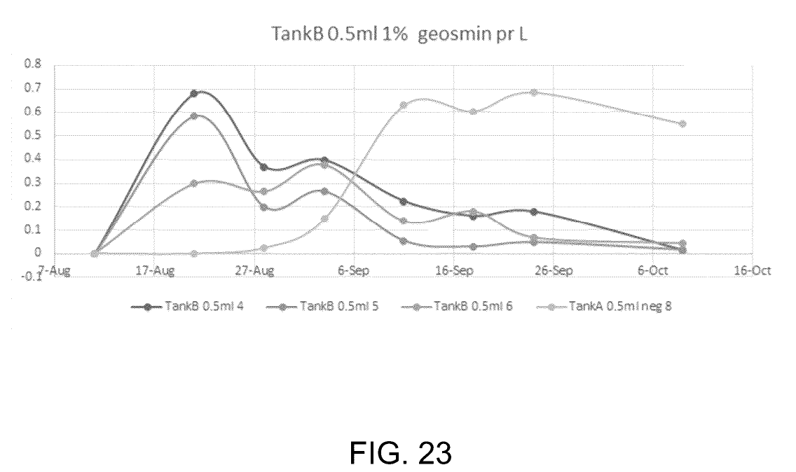

OD measurements of 20 ul 1% geosmin per L enrichments can be seen in FIG. 22. OD measurements of 1 ml 1% geosmin per L enrichments can be seen in FIG. 23.

The following samples were sent for geosmin quantification and also 16S rRNA and 18S rRNA sequencing:

TB_Start (BH water with 1 ml 1% geosmin/L), TB_4_20200821, TB_4_20200903, TB_4_20200918, TB_8n_20200821, TB_8n_20200903, TB_8n_20200918, TB_5_20200821, TB_5_20200903, TB_5_20200918—where the underlined text is the sample identified and the remaining numbers are the relevant dates.

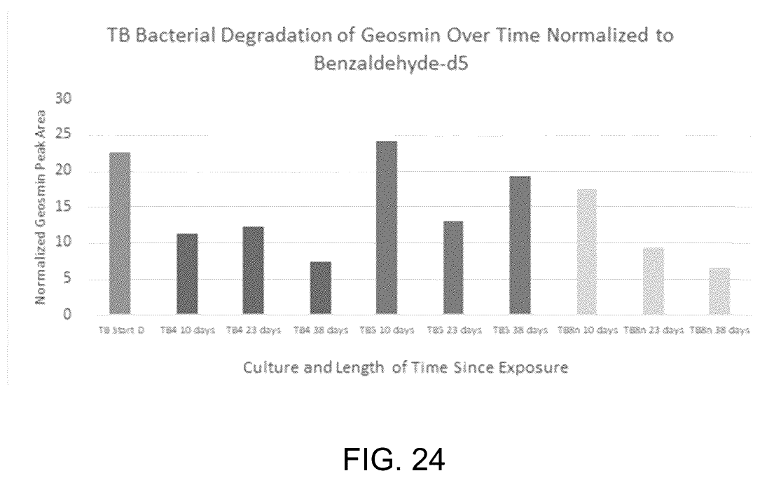

FIG. 24 shows the geosmin quantification data. The geosmin quantification of these TA and TB enrichments suggests that degradation may have happened in TB4 and the filtered water ‘control’ 8n. TB5 showed an initial decrease followed by increase in geosmin concentration, suggesting both degradation and production of geosmin.

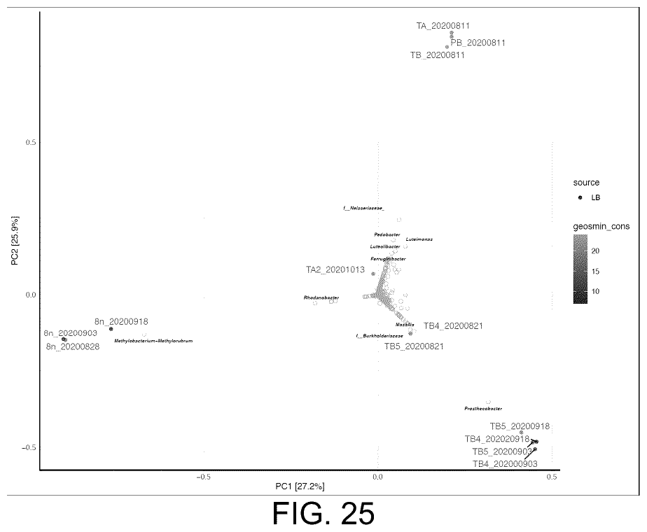

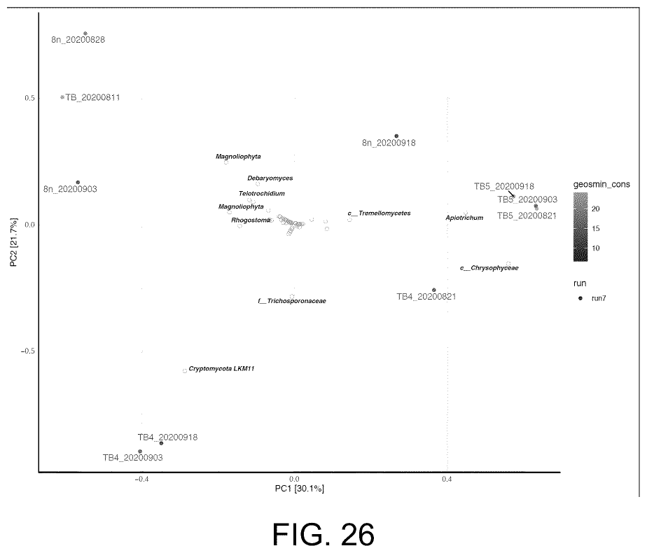

For these cultures both 16S and 18S rRNA amplicons were analysed since TB4 and TB5 looked like they had fungal growth. The 16S rRNA and 18S rRNA amplicon analyses suggested a fungi was responsible for the degradation in TB4 while a bacterial species was the most likely degrader in 8n. A PCA ordination plot of these samples is shown in FIG. 25. This PCA plot shows that the water samples from Tank A, Tank B and post bio-filter (TA_20200811, TB_20200811 and PB_20200811) were very similar and clustered distinctly from the enrichments. The filtered water enrichment 8n was distinct from the TB4 and TB5. FIG. 26 shows PCA ordination of the 18S data from the Tank A and Tank B samples. The 8n sample showed low levels of 18S rRNA reads and were dominated by plant DNA (Magnoliphyta), suggesting mostly relict DNA (DNA in the water from dead organisms) was amplified. The degrader in this community is therefore likely a prokaryotic organism.

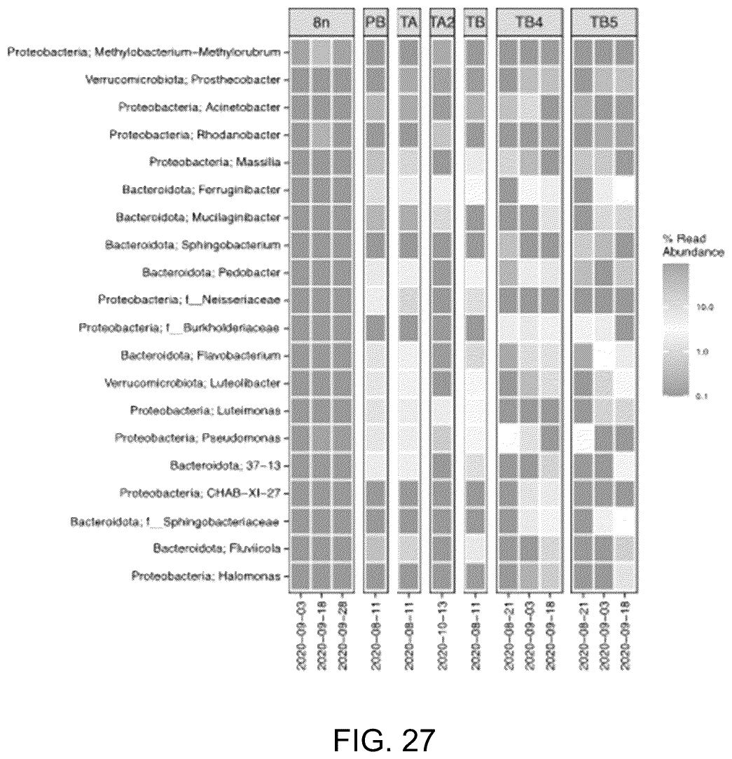

The 16S rRNA amplicon analysis of the 8n enrichment revealed that this community was characterised by high abundance of a bacterial ASV classified as Methylobacterium-Methylorubrum. These bacteria have been reported to be ‘filterable’ organisms that are detected in water filtered through 0.22 uM. They are methylotrophic bacteria characterized by the ability to grow on methanol and methylamine as sole source of carbon and energy. They also grow well on ethanol, suggesting these organisms may consume the ethanol added to the culture. These bacteria may also be directly involved in degrading geosmin since isolates of Methylobacterium has been reported to degrade geosmin. Organisms from this genus in later enrichments of other water sources were also observed (see below). This culture also contains relatively high abundance of Rhodanobacter (see heat map in FIG. 27), suggesting this may also be a degrader in this community. Rhodanobacter was also observed in the TA2 culture which received only 10 ul 1% geosmin per 500 ml water. Sequencing additional time points from this, and other 10 ul-cultures, can help determine if this is indeed the most likely degrader in these communities. For the 18S data, one yeast-type organisms, Apiotrichum, was observed. This organism was only detected at the mid time point, but it could possibly also be involved in geosmin degradation.

The TB4 and TB5 enrichments have very similar prokaryotic communities. Since they had different geosmin degradation characteristics (TB4 showed good degradation, while this was not the case for TB5 (FIG. 24)) this suggests that one of the taxa only present in TB4 is responsible for the degradation. Alternatively, TB5 might have a geosmin producer, however no obvious producer was only observed in TB5. Among bacteria, Sphingobacterium (FIG. 27) is only present at high levels in TB4 and could be a possible degrader. No geosmin degraders have been reported for this genus. However, they have been shown to degrade other recalcitrant compounds.

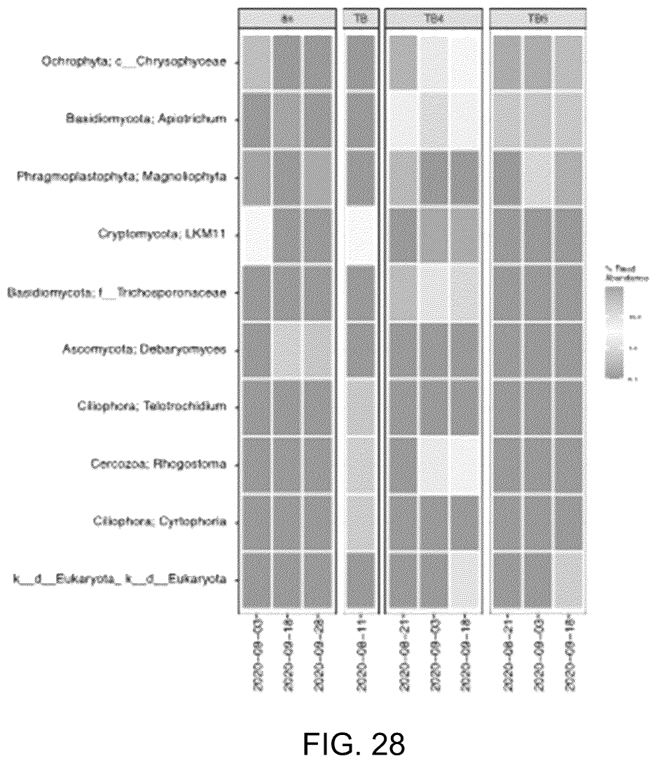

FIG. 28 is a heatmap of the ten most abundant eukaryotic genera in the Tank B water samples and enrichments established therefrom. The 18S community shows more differences between TB4 and TB5. This suggests that a eukaryotic organism might be responsible for the better performance of TB4. Two eukaryotic lineages have higher abundance in TB4 compared to TB5. Cryptomycota LKM11 are aquatic microorganisms (FIGS. 21 and 23). They are the deepest phylogenetic branch of fungi and there is little know about these organisms (Rojas-Jimenez; Lara et al., 2010, 11). The second lineage could only be determined to family level; Trichosporonaceae. This is the same family as Apiotrichum discussed above belongs to. This organism is likely the most important eukaryotic degrader in the TB4 community.

### Example 6—Further Enrichments

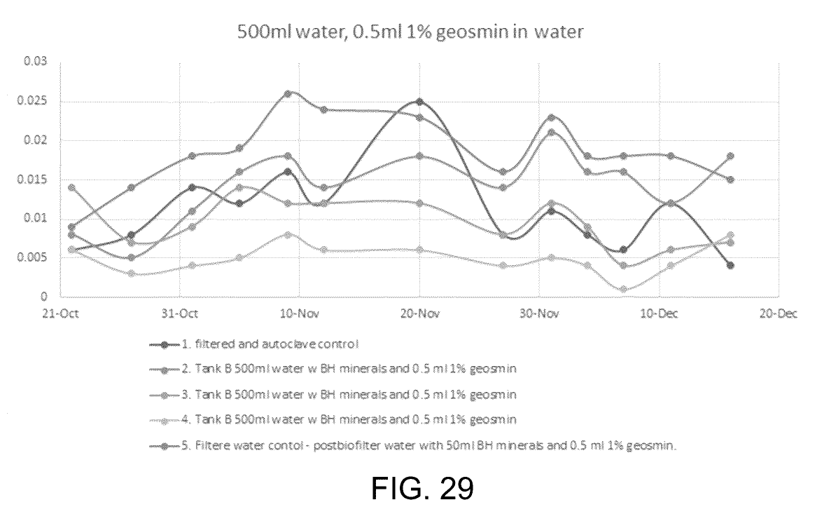

Further enrichments using BH water were made using 500 ml with 0.5 ml 1% geosmin. These were maintained and fed more geosmin approximately once a week. The OD measurements of these cultures are shown in FIG. 29.

Without being bound by any particular theory, the Examples above demonstrate that embodiments of the present disclosure can transform a sample of water that has been exposed to a target, off-flavour compound into an enriched microbiome that has the capability to degrade the target off-flavour compound, where geosmin was a specific non-limiting example of a target off-flavour compound.

All the unfiltered enrichments had high initial abundances of Acinetobacter. Since this organism also grew in the ethanol and nutrient only enrichments from BH water, it suggests that this organism is not an obligate geosmin degrader. If this bacterium is involved in degradation of geosmin, it might participate in a co-metabolic process where geosmin is broken down during growth on ethanol. Filtering the water removed Acinetobacter from the communities.

In the BH microbiomes, Gordonia and Rhodanobacter are likely degraders.

Methylobacterium and Rhodanobacter, are likely degraders in communities from Tank A or Tank B samples that were filtered. Rhodanobacter is also a likely degrader in the geosmin enrichments with low concentration of geosmin added.

A Trichosporonaceae fungi is a likely geosmin degrader in the TB4 culture.

The sequence analysis of the inoculated feed shows that the feed can be inoculated and have all the major organisms in the enrichment represented in the community.

### 2^{nd} Study: “1-Geosmin Enrichments”

TABLE 2
(“1-Geosmin enrichments”)

| | July | July | Aug | Aug | Aug | Aug | Sept | Sept | Sept | Sept |
|---|---|---|---|---|---|---|---|---|---|---|
| Enrichments | 24 | 27 | 4 | 11 | 21 | 28 | 3 | 11 | 18 | 24 |
| BH 0.5 ml | 0.306 | 0.328 | 0.580 | 0.693 | 0.544 | 0.501 | 0.492 | 0.499 | 0.169 | 0.165 |
| G 1 | | | | | | | | | | |
| BH 0.5 ml | 0.222 | 0.238 | 0.097 | 0.998 | NM | NM | NM | NM | NM | |
| G 2 | | | | | | | | | | |
| BH 0.5 ml | 0.499 | 0.510 | 0.756 | 0.196 | NM | NM | NM | NM | NM | |
| G 3 | | | | | | | | | | |
| negative | NM | 0.027 | 0.056 | 0.298 | 0.289 | 0.263 | 0.157 | 0.094 | 0.125 | 0.120 |

### 3^{rd} Study: “1-Geosmin Enrichments”

TABLE 3
(“1-Geosmin enrichments”)

| Culture | Aug 21 | Aug 28 | Sept 3 | Sept 11 | Sept 18 | Sept 24 | Oct9 |
|---|---|---|---|---|---|---|---|
| TankA 10 ul 1 | NM | 0 | 0.002 | 0 | 0.002 | 0.007 | 0.002 |
| TankA 10 ul 2 | NM | 0 | 0.004 | 0.002 | 0.003 | 0.002 | 0.001 |
| TankA 10 ul 3 | NM | 0.004 | 0.002 | 0.002 | 0.002 | 0.008 | −0.001 |
| | | | | | | | clumpy |
| TankB 0.5 ml 4 | 0.677 | 0.366 | 0.395 | 0.222 | 0.159 | 0.178 | 0.013 |
| TankB 0.5 ml 5 | 0.581 | 0.197 | 0.264 | 0.053 | 0.029 | 0.047 | 0.015 |
| TankB 0.5 ml 6 | 0.297 | 0.263 | 0.376 | 0.137 | 0.179 | 0.067 | 0.042 |
| TankA 10 ul | NM | NM | 0.001 | 0.017 | 0.019 | 0.013 | 0.009 |
| neg 7 | | | | | | | |
| TankA 0.5 ml | NM | 0.024 | 0.146 | 0.629 | 0.603 | 0.683 | 0.548 |
| neg 8 | | | | | | | |

### Example 8: Loading on Further Carrier and Cometabolic Biodegradation

Further samples (Table 4 provides the contents of these further samples) were prepared for HSSPME-GC-MS/MS analysis, using the same GC procedure as described above in Example 1.

Media mixes a-h were used to prepare these further samples as follows: diluted stock geosmin stock 10×: 100 ul geosmin in 900 ul PCR-grade water. Make medium with geosmin dilute: mix a−30 ml Thesis medium+300 ul diluted geosmin stock; mix b−30 ml Thesis medium+30 ul diluted geosmin stock, mix c−30 ml Thesis medium+3 ul diluted geosmin stock; mix d−20 ml BH medium+20 ul diluted geosmin stock; mix e−20 ml BH medium+2 ul diluted geosmin stock; mix f−20 ml mBH medium+200 ul diluted geosmin stock; mix g−20 ml mBH medium+20 ul diluted geosmin stock; and, mix h−20 ml mBH medium+2 ul diluted geosmin stock.

TABLE 4
Contents of Further Samples

| | | Start | |
|---|---|---|---|
| Name on tube: | Culture - 1 ml | concentration | |
| 1.D2fThMT1_100 ug | T#1 D2 fresh | 100 | ug/L |
| | thesis maltose | | |
| 2.D2fThMT1_10 ug | T#1 D2 fresh | 10 | ug/L |
| | thesis maltose | | |
| 3.D2fThMT1_1 ug | T#1 D2 fresh | 0.1 | ug/L |
| | thesis maltose | | |
| 4.D1fThgT1_100 ug | T#1 D1 fresh | 10 | ug/L |
| | thesis geo only | | |
| 5.D1fThgT1_10 ug | T#1 D1 fresh | 1 | ug/L |
| | thesis geo only | | |
| 6.D1fThgT1_1 ug | T#1 D1 fresh | 0.1 | ug/L |
| | thesis geo only | | |
| 7.Th_neg_100 ug | | 10 | ug/L |
| 8.Th_neg_10 ug | Data rejected due | 9 ml medium | |
| | to LOOSE LID | mix b | |
| 9.Th_neg_1 ug | Data rejected due | 9 ml medium | |
| | to LOOSE LID | mix c | |
| 10.Camph_neg_10 ug | | 10 | ug/L |
| 11.Camph_neg_1 ug | | 1 | ug/L |
| 12.ASBH_camph_10 ug | ASBH | 10 | ug/L |
| 13.ASBH_camph_1 ug | ASBH | 1 | ug/L |
| 14.D1SWmBHgT1_100 ug | D1salt T1 geo | 100 | ug/L |
| | only mBH+ | | |
| 15.D1SWmBHgT1_10 ug | D1salt T1 geo | 10 | ug/L |
| | only mBH+ | | |
| 16.D1SWmBHgT1_1 ug | D1salt T1 geo | 1 | ug/L |
| | only mBH+ | | |
| 17.mBHneg_100 ug | | 100 | ug/L |
| 18.mBHneg_10 ug | | 10 | ug/L |
| 19. mBHneg_1 ug | | 1 | ug/L |

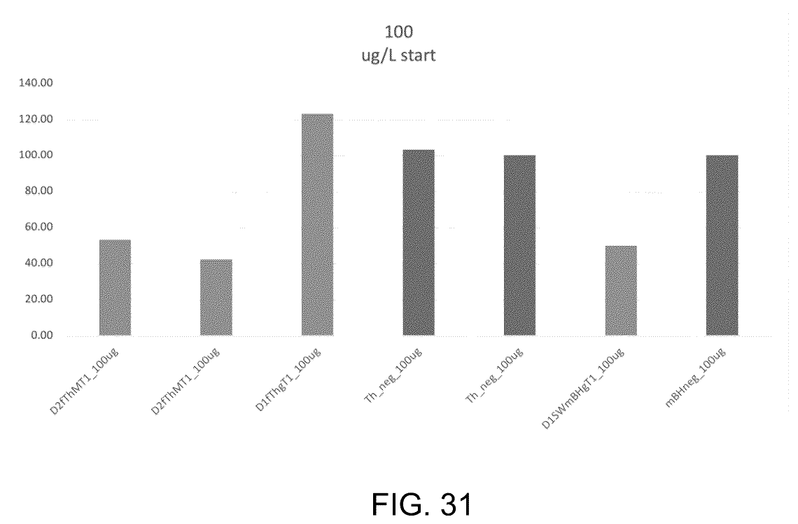

FIG. 31 shows the detected levels of geosmin in the samples that were treated with 100 μg/L of geosmin. Two microbiomes demonstrated lower levels of geosmin detected: D2fThMT1 (Maltose) and D1SWmBHgT1 (geosmin only).

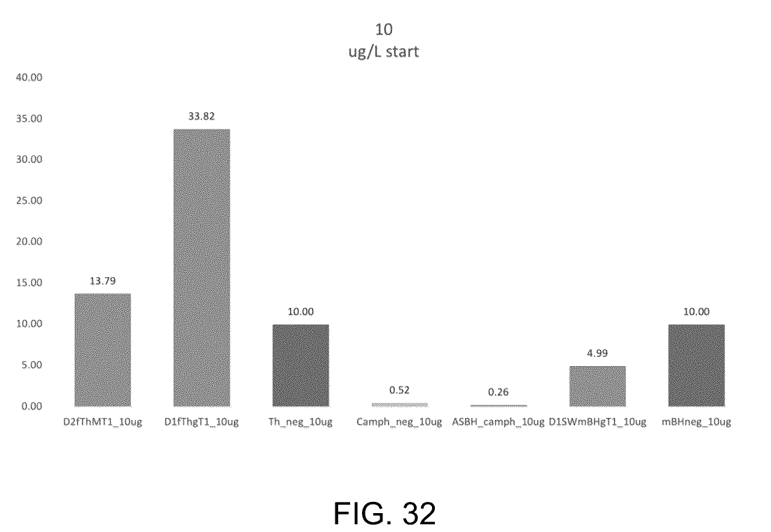

FIG. 32 shows the detected levels of geosmin in the samples that were treated with 10 μg/L of geosmin. Two microbiomes demonstrated lower levels of geosmin: ASBH+camph (camphor used as carrier), D1SWmBHgT1 (geosmin only), and Camphor alone.

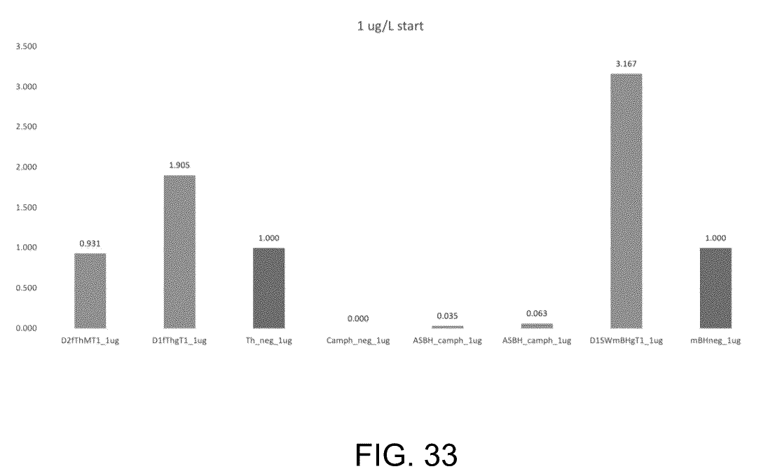

FIG. 33 shows the detected levels of geosmin in the samples that were treated with 1 μg/L of geosmin. In these two further samples, two enriched microbiomes demonstrated 50% decrease in detected levels the geosmin: D2fThMT1 and D1SWmBHgT. D2fThMT1 is a freshwater microbiome and D1SWmBHgT1 is a saltwater/marine microbiome. D2fThMT1, which was grown on maltose, did not degrade geosmin at the 10 μg/L concentration. Without being bound by any particular theory, this result may be due to the geosmin degrading enriched microbiomes being out-competed at the lower concentrations and indicates that this may not act as a co-metabolic microbiome at these concentrations of geosmin.

These results also demonstrate the effect of adding camphor to the enriched microbiomes to act as a carrier. The AS-BH enriched microbiome was used, which also demonstrated about 50% decrease in geosmin levels at treatment concentrations from 1-100 μg/L, if sufficient biomass is added (˜1 ml of enriched microbiome in about 10 ml). At 10 μg/L of geosmin treatment, about 95% of the geosmin was removed with camphor only and 97% when camphor was used as a carrier for the enriched microbiome. Using a starting concentration of 1 μg/L geosmin resulted in undetectable levels of geosmin when camphor was used as a carrier.

### Example 9: Investigation into Geosmin Degradation with Combination of Enriched Microbial Consortium and Carrier

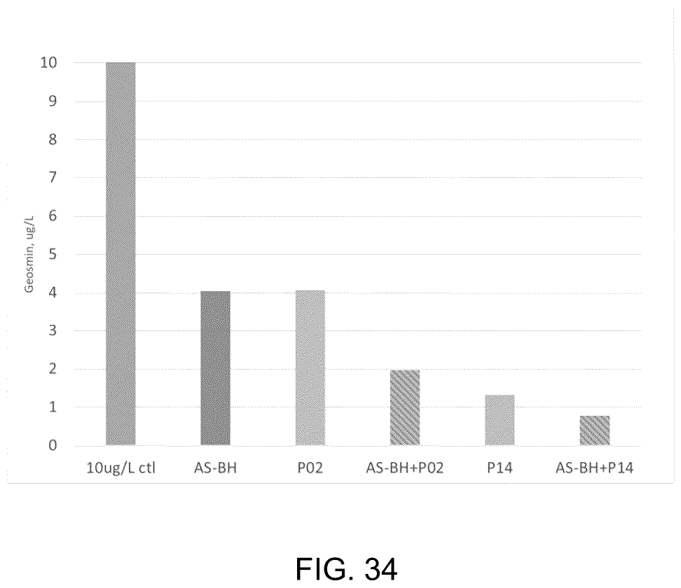

FIG. 34 shows geosmin degradation data when the enriched microbial consortium AS-BH was inoculated, via its biofilm, on to one of two carriers: sterile paraffin beads (referred to as P02) and non-sterile paraffin beads (referred to as P14), where the P14 carrier had a higher surface area:volume ratio as compared to the P02 carrier.

The trial was run for 14 days and the quantification data revealed that the AS-BH consortium removed approximately 60% of the geosmin present. Carrier P02 without biofilm removed similar amounts. Whereas when the P02 carrier was inoculated with an AS-BH biofilm a higher removal (about 80%) of geosmin than either the carrier or the enriched microbial consortium alone was observed.

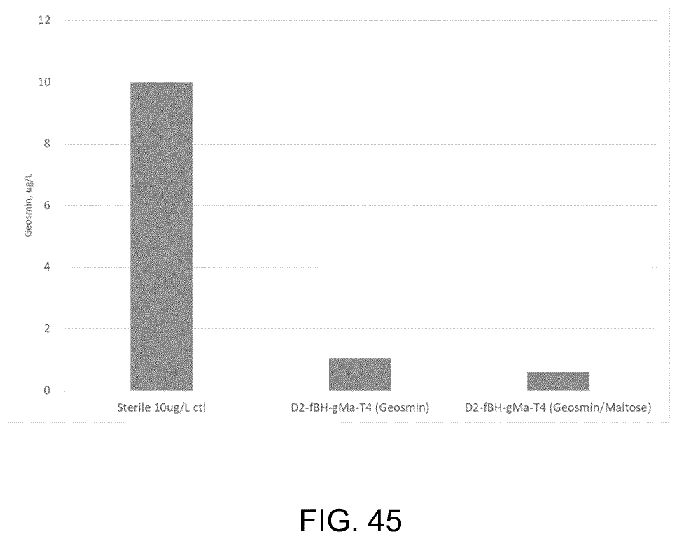

The P14 carrier alone, without biofilm, removed about 85% of the geosmin. Once the P14 carrier was inoculated with the AS-BH biofilm, the resulting combination of the P14 carrier and the AS-BH consortium biofilm removed greater than 90% of the geosmin. Without being bound by any particular theory, the results shown in FIG. 45 may support using a combination of activated microbiome consortium and a carrier to reduce or remove the amount of geosmin present in water, including large amounts of geosmin.

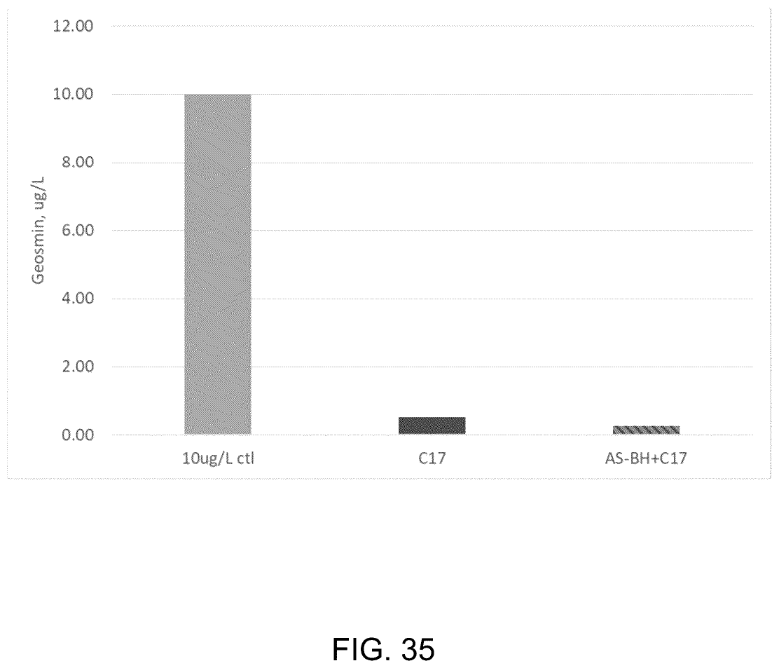

FIG. 35 shows geosmin degradation data when the enriched microbial consortium AS-BH was inoculated, via its biofilm, on to a further abiotic carrier consisting of camphor beads (referred to as carrier C17).

The trial was run for 14 days and quantification data reveals that carrier C17 without being inoculated with the AS-BH biofilm removed approximately 90% of detectable geosmin. Whereas, inoculating the C17 carrier with an AS-BH biofilm resulted in a greater higher removal, greater than about 95%, of geosmin which was higher than both of the C17 carrier alone or the AS-BH consortium alone. Without being bound by any particular theory, this data may further support using different carriers as part of an enriched microbial consortium carrier combination to reduce or remove the amount of geosmin present in water, including large amounts of geosmin.

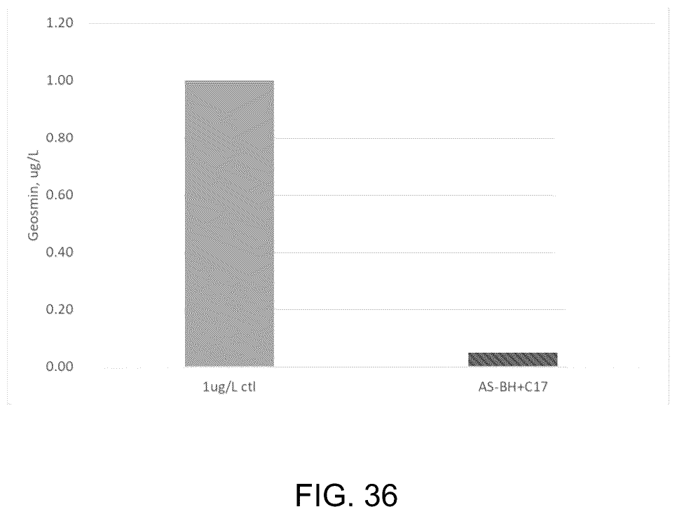

FIG. 36 shows further geosmin degradation data when the enriched microbial consortium AS-BH was inoculated, via its biofilm, on to the C17 carrier in the presence of a 1 μg/L geosmin challenge. The trial was run for 14 days, and two replicates were performed. Quantification of the data reveals that C17 carrier inoculated with AS-BH biofilm removed more than 95% of the low amount of geosmin present, resulting in a final concentration of approximately 50 ng/L, within the range of human detection. For context, the 1 μg/L challenge represents an amount of the off-flavour compound that a human would detect and find the flavour very off.

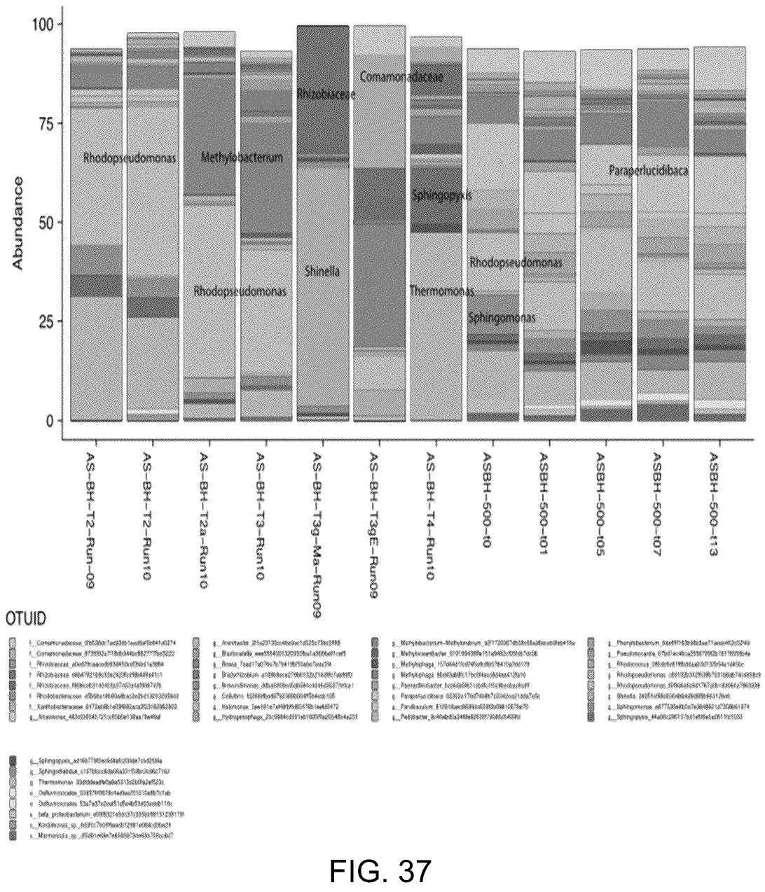

FIG. 37 shows the data from the multiple amplicon sequencing analyses on subsamples of the AS-BH consortium that were performed to determine the constituent members of the enriched microbial consortium AS-BH and to identify probable degrading members. T2, T3, and T4 columns are transfer cultures, propagations of the original consortium exposed to different conditions. T2 and T3 cultures have been fed exclusively geosmin, and contain high abundances of Rhodopseudomonas, Thermomonas and Methylobacterium-Methylorubrum. T3g, T3gE and T4 transfers are grown on supplemental carbon sources, which change their community composition, but increases biomass. The right half of the figure, with ASBH-500 cultures, are samples taken during the time series experiment described above. This scaled-up consortium has higher diversity and high abundance of Paraperlucidibaca. This culture was fed a lower amount of geosmin during the time series experiment (20 μg/L vs. 100 μg/L) and the higher diversity might reflect this, where a lower initial geosmin concentration reflects in a lower stimulation of the degrader and increased relative abundance of the supporting community. These diverse communities, nonetheless, are effect geosmin degraders as indicated by the time-series analysis. Rhodopseudomonas is the most likely degrader. Paraperlucidibaca is a hydrocarbon degrader and could also be involved in geosmin degradation.

### Example 10: Investigation into Geosmin Degradation and Stereo-Specificity

Some target compounds may have one or more chiral centers and, therefore, they may have stereospecific enantiomers with the same organization of chemical elements but the mirror image of each other. Enantiomers, which may also be referred to as optical isomers, stereoisomers and optical antipodes, differ in their respective optical activity but display identical physical and chemical properties. However, enantiomers are known to have the potential for different biological activities.

There are known methods to classify the stereo-chemistry of a compound. L and D is based on optical rotation and R/S is based on the chiral center. Enantiomers capable of rotating polarized light clockwise and anticlockwise are labeled as (+) and (−), respectively. Depending on the compound an (R)-enantiomer can be either (+)- or (−)-isomer. Geosmin exists as (+) and (−) enantiomers.

Odor outbreaks are caused by biological production of the naturally occurring (−)-enantiomers of geosmin, which are known to be about 10 times more potent than the (+) enantiomers of geosmin.

Enantiomer-specificity of enzymes is well known. Based on observations from prior testing that most micro-organism cultures degrade about 50% of the geosmin added for these experiments, it was considered whether the cultures contain microorganisms with enzymes with stereo-specificity for (−)-geosmin. The enzymes of the microorganisms could specifically target (−)-geosmin, again, the naturally produced enantiomer (or optical stereoisomer).

Accordingly, degradation experiments using pure (−)-geosmin were performed.

The (−)-geosmin was obtained from MuseChem™ (available commercially). Previous examples described herein used a mix of +/−geosmin (commercially available from Sigma Aldrich™. Quantification of geosmin was performed using HSSPME-GC-MS/MS.

Several degradation experiments on prototype degrading microbial consortia are described herein below. The microbial consortium has been stably maintained with solely geosmin as a carbon source (or geosmin with one or more further metabolic substrates) for over a year as well as other degrading consortia produced from different communities.

The enriched microbial consortium that has been maintained the longest, referred to as AS-BH in the examples below, was based on a community collected from the same commercial RAS, AS, described in Example 1. BH here refers to Buschnell-Haas medium. The long-term stability of this microbial consortium, and the various experiments carried out over time, are demonstrative of this culture's ability to consistently degrade geosmin.

The AS-BH enriched microbial consortium was consistently able to remove 40-60% of the waterborne geosmin it was fed. This was achieved across different starting concentrations (100 μg/L, 10 μg/L, 1 μg/L and 0.1 μg/L) in multiple replicates.

Six microbial community cultures were used in the tests of this Example. Namely, AS-BH-T2500, AS-BH-T3-R, AS-BH-R-T4, D2fBHgMaT7, D1-salt-geo-mBH-T4 and D2-s-mBH-gM-T5. AS-BH-T2-500 is a scale-up of an older culture (ASBH), and has been growing for at least 1 year. AS-BH-T3-R and AS-BH-R-T4 are revival of frozen glycerol stock of AS-BH-T2-500 and a transfer of the revival culture, respectively. Each culture was spun down in 3 tubes with 1.5 ml culture to remove old medium, and the pellet was re-suspended in about 500 ml of medium. D2fBHgMaT7 was scaled up to 500 ml and was fed both maltose and geosmin.

The three amounts of (−)-geosmin were tested: 100 μg/L, 10 μg/L and 1 μg/L using the following microbiome mixes:
1. Mix A—100 μg/L in BH medium: 50 ml BH medium+500 ul diluted gesomin
2. Mix B—10 μg/L in BH medium: 50 ml BH medium+50 ul diluted gesomin
3. Mix C—1 μg/L in BH medium: 50 ml BH medium+5 ul diluted gesomin
4. Mix D—100 μg/L in marine BH medium: 30 ml mBH medium+300 ul diluted gesomin
5. Mix E—10 μg/L in marine BH medium: 30 ml mBH medium+30 ul diluted gesomin
6. Mix F—1 μg/L in marine BH medium: 30 ml mBH medium+3 ul diluted gesomin

Table 1 below sets out the parameters of how the (−)-geosmin degrading activity of the various mixtures was assessed.

TABLE 1
Experimental parameters for (−)-geosmin degradation assessment.

| | Name and | |
|---|---|---|
| Tube | (−)-geosmin | |
| no | challenge amount | Culture + mix |
| 1 | BH-100 ug | 9.5 ml Mix A |
| 2 | ASBH-500-100 ug | 9.5 ml mix A, 500 ul ASBH-500 culture re-suspended |
| 3 | AS-BH-T3-R-100 ug | 9.5 ml mix A, 500 ul AS-BH-T3-R culture re-suspended |
| 4 | AS-BH-R-T4-100 ug | 9.5 ml mix A, 500 ul AS-BH-R-T4 culture re-suspended |
| 5 | D2fBHgMaT4-100 ug | 9.5 ml mix A, 500 ul D2fBHgMaT4 culture re-suspended, |
| | | 100 ul 1% maltose |
| 6 | BH-10 ug | 9.5 ml Mix B |
| 7 | ASBH-500-10 ug | 9.5 ml mix B, 500 ul ASBH-500 culture re-suspended |
| 8 | AS-BH-T3-R-10 ug | 9.5 ml mix B, 500 ul AS-BH-T3-R culture re-suspended |
| 9 | AS-BH-R-T4-10 ug | 9.5 ml mix B, 500 ul AS-BH-R-T4 culture re-suspended |
| 10 | D2fBHgMaT4-10 ug | 9.5 ml mix B, 500 ul D2fBHgMaT4 culture re-suspended, |
| | | 100 ul 1% maltose |
| 11 | BH-1 ug | 9.5 ml Mix C |
| 12 | ASBH-500-1 ug | 9.5 ml mix C, 500 ul ASBH-500 culture re-suspended |
| 13 | AS-BH-T3-R-1 ug | 9.5 ml mix C, 500 ul AS-BH-T3-R culture re-suspended |
| 14 | AS-BH-R-T4-1 ug | 9.5 ml mix C, 500 ul AS-BH-R-T4 culture re-suspended |
| 15 | D2fBHgMaT4-1 ug | 9.5 ml mix C, 500 ul D2fBHgMaT4 culture re-suspended, |
| | | 100 ul 1% maltose |
| 16 | mBH-100 ug | 9.5 ml Mix D |
| 17 | D1-salt-geo-mBH-T4-100 ug | 9.5 ml mix D, 500 ul D1-salt-geo-mBH-T4 re-suspended |
| 18 | D2-s-mBH-gM-T5-100 ug | 9.5 ml mix D, 500 ul D2-s-mBH-gM-T5 culture |
| | | re-suspended, 100 ul 1% maltose |
| 19 | mBH-10 ug | 9.5 ml Mix E |
| 20 | D1-salt-geo-mBH-T4-10 ug | 9.5 ml mix E, 500 ul D1-salt-geo-mBH-T4 re-suspended |
| 21 | D2-s-mBH-gM-T5-10 ug | 9.5 ml mix E, 500 ul D2-s-mBH-gM-T5 culture |
| | | re-suspended, 100 ul 1% maltose |
| 22 | mBH-1 ug | 9.5 ml Mix F |
| 23 | D1-salt-geo-mBH-T4-1 ug | 9.5 ml mix F, 500 ul D1-salt-geo-mBH-T4 re-suspended |
| 24 | D2-s-mBH-gM-T5-1 ug | 9.5 ml mix F, 500 ul D2-s-mBH-gM-T5 culture |
| | | re-suspended, 100 ul 1% maltose |

Each tube was sealed with parafilm and incubated upside down on a shaker for about two weeks. A time 0 sample was also frozen for comparison.

In general, the results from these degradation tests revealed at least a 10-fold lower (−)-geosmin concentration in the tubes with cultures compared to the sterile controls.

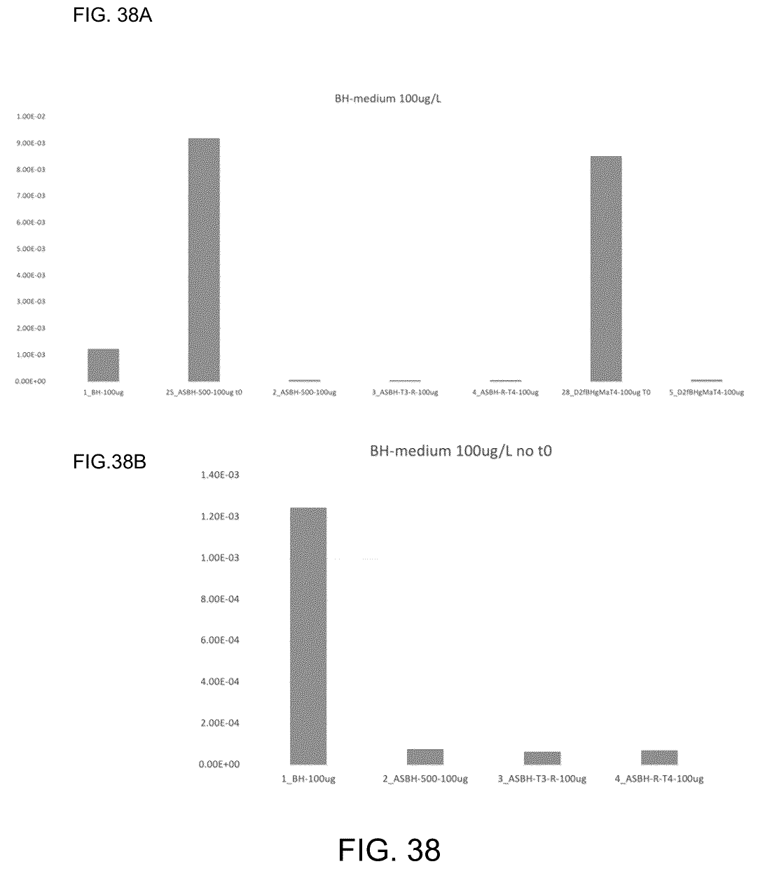

The results in FIG. 38A show that the highest (−)-geosmin concentrations were observed in the two TO samples in the tubes with 100 μg/L of (−)-geosmin. The sterile control (the 1_BH-100 g sample) also shows lower amounts than these samples, which could indicate loss by evaporation in this sample. This possibility can be excluded for the other samples, so the results were plotted in FIG. 38B excluding the TO samples. Samples 25 and 28 are frozen samples from TO. This show significantly lower concentrations in all the cultures also when compared to the sterile control (1_BH-100 g).

It was assumed that all the tubes had lost substantially the same amount as the sterile control, due to evaporation, then about 94-95% of the (−)-geosmin was consumed by the microbial community.

Similar results were obtained for the cultures with 10 μg/L of (−)-geosmin. For these samples there were no frozen t0 samples, however, compared to the sterile control, the microbial community removed 98-97% of the (−)-geosmin. This means that if there was no loss in the sterile control (i.e. sample 1 corresponds to 10 μg/L), then there was about 300 ng left in the AS-BH-R-T4-10 μg culture (which has the most left).

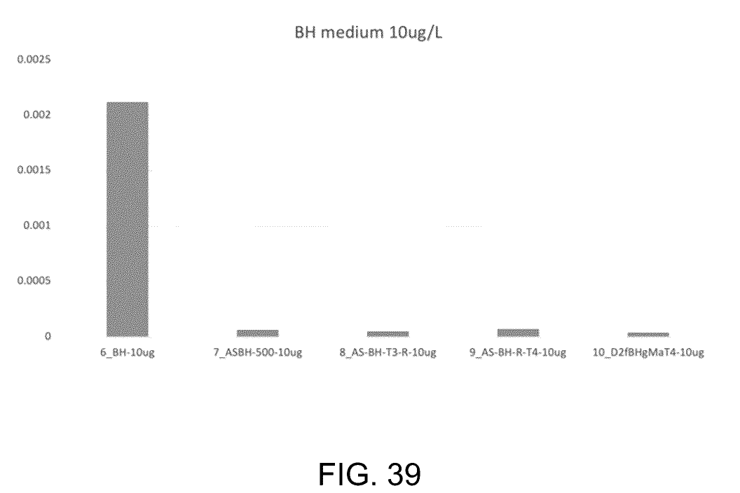

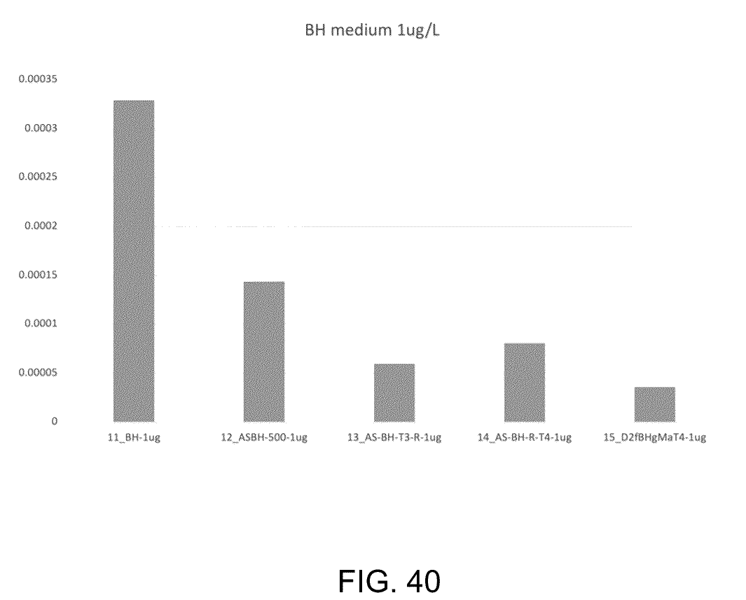

FIG. 39 shows the data where cultures were added to BH-medium with 10 μg/L of (−)-geosmin. Sample 6 is the sterile control. FIG. 40 shows the data from cultures added to BH-medium with 1 μg/L of (−)-geosmin. Sample 11 is the sterile control. There were lower relative amounts of loss from the 1 μg/L samples (FIG. 40). This might partly be due to carry-over of (+)-geosmin for some of the cultures since there was likely accumulation of the (+) enantiomer over the time the cultures have been maintained. Such carry-over may show up more significantly at these lower concentrations. In agreement with this, ASBH-T2-500 is the oldest culture tested and has 43% of the geosmin left (compared to the sterile control). It is therefore likely that washing the cell pellet did not remove of all the (+)-geosmin.

Two marine cultures (mBH) were tested—also here there was one (−)-geosmin-only culture and one culture fed both (−)-geosmin and maltose:
D1-salt-geo-mBH-T4
D2-s-mBH-gMa-T5

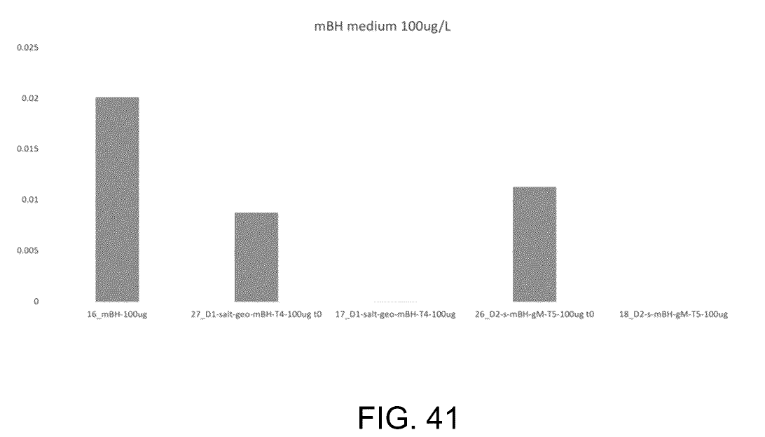

For the marine water samples, the highest amounts of (−)-geosmin were observed in the sterile control. 99% removal of (−)-geosmin in the cultures was also observed compared to either the sterile control or the t0 samples. FIG. 41 shows the data where the cultures were added to marine BH-medium with 100 μg/L (−)-geosmin samples. Sample 16 is the sterile control. 27 and 26 are the to samples. Less than 1% of the geosmin is detected in samples 17 and 18 but it appears to be zero because of scale of y-axis.

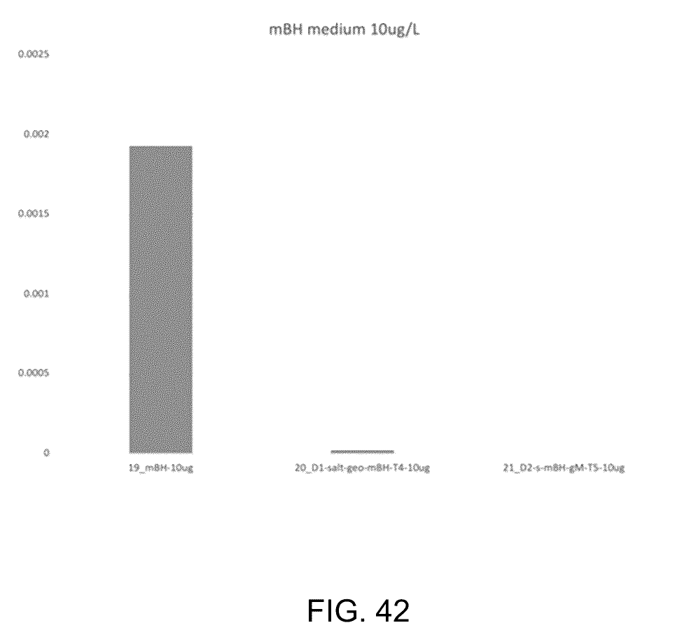

FIG. 42 shows the data from marine BH-medium with 10 ug/L of (−)-geosmin. Sample 16 is the sterile control. At 10 ug/L no geosmin was detected in sample 21, suggesting that the D2-s-mBH-gMa-T5 (with maltose) removed substantially all the (−)-geosmin present. For D1-salt-geo-mBH-T4 there was about 100 ng/L remaining.

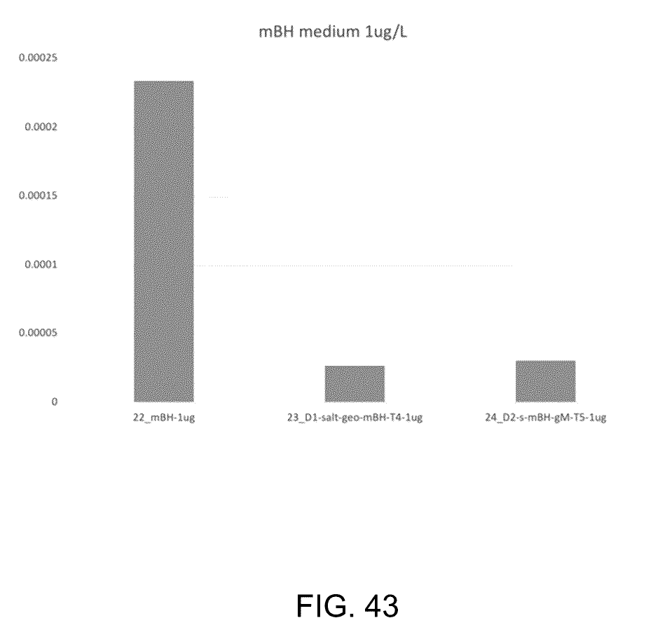

FIG. 43 shows the data from marine BH-medium with 1 ug/L of (−)-geosmin. Sample 22 is the sterile control. As observed for the freshwater cultures, there was lower relative degradation in the samples with 1 ug/L geosmin. Here the microbial community has removed ˜90% of the geosmin. It is likely that some of this could be carry-over of (+)-geosmin from maintaining the culture with a racemic mixture in the past.

Without being bound to any particular theory, the cultures removed about 94-100% of the (−)-geosmin at high concentrations (10-100 μg/L). Lower removal was seen at those cultures added to BH medium with 1 μg/L of (−)-geosmin. However, these results are likely confounded by the fact that there may be some carry-over of (+)-geosmin, which will more significantly affect the measurements at the lowest concentrations. It is likely that washing the cells pellets did remove not all of the (+)-geosmin, which is supported by the fact that the most geosmin was observed in the oldest cultures. Alternatively, degradation might be less effective at lower concentrations-which could support the need to up-concentrate the (−)-geosmin in the bioreactor using a hydrophobic material such as paraffin or alginate beads with or without vegetable oil inclusions.

The cultures fed maltose appeared to remove more (−)-geosmin, as compared to cultures grown with (−)-geosmin only. This observation was made in both freshwater and marine samples. Without being bound by any particular theory, supplying a further metabolic substrate to the microbiomes, such as a further source of carbon like maltose or other microbiome metabolic substrates, may result in higher cell density within the microbiome and, therefore, may support improved (−)-geosmin removal.

The results reported above demonstrate that the embodiments of the present disclosure can remove all, or substantially all, of the (−)-geosmin added. This outcome suggests that the enriched microbiomes comprise organisms with enzymes that can target (−)-geosmin specifically.

For both amplicon and metagenome analyses, both the Illumina short-read second-generation sequencing and Oxford Nanopore long-read third-generation sequencing methodologies were used. All data were analysed using open-source containerized bioinformatics pipelines (e.g. QIIME2, Nanoclust, Emu, kaiju, MetaErg and others), which have been customized for the specific uses described herein.

By performing whole metagenome sequencing, it was possible to examine the genes and metabolic pathways contained in the enriched microbial consortium, and determine the genetic traits—or trends in genetic traits—that allow the enriched microbial consortium to degrade off-flavour compounds, such as geosmin. The analysis was performed on one subculture of the AS-BH consortium, generating sufficient data to allow for the detection and analysis of constituent microbial members of the AS-BH consortium that make up >1% of the total population. Taxonomy of the reads was assessed using Kaiju at KBase, showing high abundances of Methylorubrum and Rhodopseudomonas, candidate degraders from 16S analysis. Annotation data from the metagenome reveals a high abundance of monooxygenase-associated gene(s), as well as the presence of a limonene hydroxylase gene with known terpene-degrading capacity, a likely enzymatic candidate for geosmin removal activity.

Without being bound by any particular theory, discovery of these enzymatic genes within the AS-BH consortium may validate the ‘degrading consortium’ concept. For example, native Rhodopseudomonas does not typically produce limonene hydroxylase and no single strain of bacteria would carry the full complement of monooxygenases observed. An enriched microbial consortium, such as the AS-BH consortium, may be able to deliver greater genetic functionality to a treatment environment.

A generally accepted threshold for human detection of geosmin is in the range of about 20 to about 50 ng/L. Degradation experiments from a 0.1 μg/L (i.e. 100 ng/L) starting point were able to achieve greater than about 50% removal of geosmin, resulting in final concentrations that fall into or below this detectable range. Without being bound by any particular theory, the embodiments of the present disclosure may be capable of reducing the amounts of target compounds, such as geosmin and other off-flavour compounds, within RAS water to levels at or below the detectable range for humans.

As described further above, some embodiments of the present disclosure relate to combining the activated microbial consortia that can degrade the off-flavour compounds with application-specific carriers. Using these combinations of activated microbial consortia and carriers may allow for optimal capture and up-concentration of the off-flavour compound to facilitate effective and continuous degradation of off-flavour compound by the activated microbial consortia. Several carriers and substrates have been tested to date, in an effort to determine a suitable combination for RAS applications, including two categories of an abiotic carrier that is available in a bead form and discussed further below. The combination of the enriched microbial consortium and the carrier appears to enhance the ability of the enriched microbial consortium to degrade off-flavour compounds, such as geosmin, resulting in a greater percentage of off-flavour compound removal than either the enriched microbial consortium or the carrier alone.

Scalability is a potential issue that relates to the use of activated microbial consortia to reduce the amount of target compounds, such as off-flavour compounds, within RAS water to desired levels. One strategy has been to target potential co-metabolic biodegraders. For example, activated microbial consortia that are able to degrade both pure geosmin as well as geosmin supplemented with a metabolic substrate, such as one or more additional carbon sources, could be used in a production environment to accelerate culture growth, while maintaining geosmin-targeting activity. Experimentation with various additional substrates such as maltose (malt sugar) are described below. Further examples of additional substrates may include ethanol, ammonium, sucrose, yeast, methanol and combinations thereof.

### Example 12: Investigation into Geosmin Degradation with Co-Metabolic Substrates and Enriched Microbial Consortium

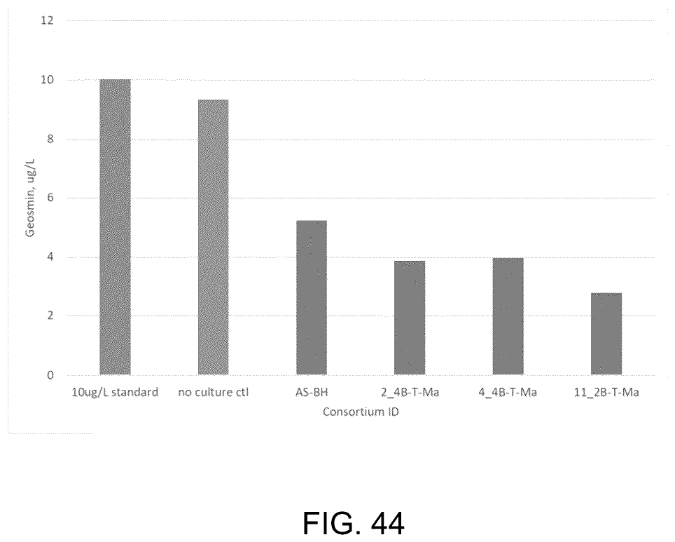

FIG. 44 shows further data that demonstrates geosmin degradation by an enriched geosmin degrading consortia that was provided a substrate for metabolism where such metabolism is not directly related to geosmin metabolism, which may also be referred to as unrelated metabolic target compound or a substrate.

This trial was run for 7 days, and quantification data reveals that consortia 2_4B-T-Ma, 4_4B-T-Ma, and 11_2B-T-Ma are capable of degrading >60% 10 μg/L geosmin. The “Ma” cultures are grown with both geosmin and maltose added. These show higher growth rates than geosmin-alone cultures, and comparable or improved geosmin degradation rates than the first-generation AS-BH consortium. Without being bound by any particular theory, these results may support using a substrate, such as maltose, for generating a geosmin-degrading activated microbial consortia.

FIG. 45 shows further geosmin degradation data when the enriched microbial consortium was grown with maltose and geosmin (D2-fBH-gMA-T4) that was derived from the AS commercial RAS facility. Geosmin degradation performance was then tested by isolating the consortium, and presenting it with either geosmin alone (middle column) or a combination of geosmin and maltose (right column).

The trial was run for 7 days, and quantification of the data reveals that greater than a 90% removal of the geosmin in the geosmin-only condition, and more than 95% removal of geosmin in the geosmin and maltose condition.

Without being bound by any particular theory, the examples described here may support the use of an enriched microbial consortium that is activated with an off-flavour compound and a further metabolic substrate. Furthermore, the biofilm of this co-metabolic enriched microbial consortium may be used to inoculate a carrier for enhanced reducing or removal of off-flavour compounds from water.

1. A composition for deploying into water of a recirculating aquaculture system (RAS), the composition comprising:
(a) an enriched microbiome that is configured to reduce an amount of a target compound by a desired amount within the water; and
(b) a carrier that is configured to induce the enriched microbiome to produce a biofilm or to sequester at least a portion of the target compound from the water or both.

2. The composition of claim 1, wherein the target compound is a terpene, a terpenoid, an amine-based compound, a haloanisole, a steroid or combinations thereof.

3. The composition of claim 1, wherein the target compound is geosmin, 2-methylisoborneol, cortisol or combinations thereof.

4. The composition of claim 1, wherein the carrier is a particle that is at least partially suspendible in the water.

5. The composition of claim 1, wherein the carrier is a material that is a fixed component, a constructed component or an installed component of the RAS.

6. The composition of claim 5, wherein the fixed component, the constructed component or the installed component of the RAS includes one or more of a filter, a tank liner, a conduit liner or combinations thereof.

7. The composition of claim 1, wherein the carrier sequesters at least a portion of the target compound from the water by adsorbing or absorbing the target compound from the water.

8. The composition of claim 1, wherein the carrier is one or more of a waxy carrier; a fresh water metaoza, a saltwater metazoa; a hydrogel; an alginate; a moving bed reactor substrate; a mineral-based insulation; fish feed; or a clay.

9. A method for preparing a microbiome-based composition for deploying into water of a RAS, the method comprising:
(a) selecting a microbiome sample from a water source that has been exposed to a target compound;
(b) preparing an inoculum from the microbiome sample;
(c) enriching the inoculum to produce a microbial consortium that degrades, consumes or otherwise converts one or more target compounds in the inoculum by culturing the inoculum in a medium that contains the one or more target compounds to produce an enriched microbial consortium;
(d) culturing the enriched microbial consortium in a suitable nutrient medium for producing greater quantities of the enriched microbial consortium.

10. A method for preparing a microbiome-based composition for deploying into water of a RAS, the method comprising:
(a) selecting a microbiome sample from a water source that has been exposed to an unrelated metabolic target compound, wherein metabolizing of the unrelated metabolic target compound results in degradation of a target compound;
(b) preparing an inoculum from the microbiome sample;
(c) enriching the inoculum to produce an enriched microbial consortium that degrades, consumes or otherwise converts one or more target, off-flavour compounds in the inoculum by culturing the inoculum in a medium that contains the one or more target, off-flavour compounds, the unrelated metabolic target compound or both to produce an enriched microbial consortium;
(d) culturing the enriched microbial consortium in a suitable nutrient medium for producing greater quantities of microbial consortium.

11. The method of claim 9 or claim 10, wherein the target compound is a terpene, a terpenoid, an amine-based compound, a haloanisole, a steroid or combinations thereof.

12. The composition of claim 9 or claim 10, wherein the target compounds is geosmin, 2-methylisoborneol, cortisol or combinations thereof.

13. The composition of claim 9 or claim 10, wherein the medium contains geosmin, 2-methylisoborneol, cortisol or combinations thereof.

14. The method of claim 9 or claim 10, further comprising a step of mixing the plurality of enriched microbial consortium with a selected carrier to produce microbiome aggregates.

15. A filter for use with a recirculating aquaculture system, the filter comprising:
(a) a surface;
(b) a microbiome aggregate adhered to the surface, wherein the microbiome aggregate comprises an enriched microbial consortium in a suitable nutrient medium for producing greater quantities of microbial consortium; and
(c) a biofilm and wherein the enriched microbial consortium is configured to reduce an amount of a target compound within water that passes along the surface.

16. The filter of claim 15, wherein the target compound is a terpene, a terpenoid, an amine-based compound, a haloanisole, a steroid or combinations thereof.

17. The filter of claim 15, wherein the target compound is geosmin, 2-methylisoborneol, cortisol or combinations thereof.

18. The filter of any one of claims 15-17, wherein the filter is further configured to remove solids from the water.

19. The filter of any one of claims 15-17, wherein the filter is further configured to reduce the level of one or more chemicals in the water.

20. A recirculating aquaculture system (RAS) comprising:
(a) a holding tank for holding a body of water;
(b) a waste removal system that is configured to reduce one or both of chemical waste or solid waste within the body of water;
(c) one or more conduits for fluidly connecting the holding tank and the waste removal system;
(d) a pumping system for circulating water through the one or more conduits; and
(e) a microbiome aggregate that comprises an enriched microbial consortium adhered to a surface of a carrier by a biofilm and wherein the enriched microbial consortium is configured to reduce an amount of a target compound within body of water.

21. The RAS of claim 20, wherein the carrier is configured to induce the enriched microbial consortium to produce the biofilm, to sequester at least a portion of the target compound from the water or both.

22. The RAS of claim 21, wherein the carrier sequesters at least a portion of the target compound by adsorbing or absorbing the target compound from the water.

23. The composition of claim 21, wherein the carrier is a fixed, constructed or installed component of the RAS that includes one or more of a filter, a tank liner, a conduit liner or combinations thereof.

24. The RAS of claim 20, wherein the carrier is a particle that is at least partially suspendible in the water.
